(19) United States
(12) Patent Application Publication
(10) Pub. No.: US 2024/0417350 A1
(43) Pub. Date: Dec. 19, 2024

(54) PROCESSES FOR THE INTEGRATION OF HYDROLYSIS OF RENEWABLE GLYCERIDES WITH THE GENERATION OF PARAFFINS AND PROPYLENE GLYCOL

(71) Applicant: AYAS Renewables, Inc., Tuscaloosa, AL (US)

(72) Inventors: Mark G. TEGEN, Austin, TX (US); William Rusty SUTTERLIN, Hoover, AL (US)

(21) Appl. No.: 18/701,404

(22) PCT Filed: Oct. 14, 2022
(86) PCT No.: PCT/US2022/046688

§ 371 Date: Apr. 15, 2024

Related U.S. Application Data
(60) Provisional application No. 63/256,449, filed on Oct. 15, 2021.

(51) Int. Cl. C07C 29/09; B01J 19/24; C07C 1/213; C07C 5/27

(52) U.S. Cl. CPC C07C 29/095; B01J 19/2445; B01J 19/245; C07C 1/213; C07C 5/27

(57) ABSTRACT

In alternative embodiments, provided processes for the integration of hydrolysis of renewable glycerides for the subsequent generation of paraffins (or a mixture of saturated hydrocarbons, or a mixture of alkanes containing around 80% to 90% linear chains (n-paraffin) with about 20 to 30 carbons in length) and propylene glycol. In alternative embodiments, provided is an improved and integrated process for producing paraffins and propylene glycol from renewable feedstocks comprising first subjecting renewable glycerides to a hydrolysis process step to generate free fatty acids (FFAs) and glycerol. The generated FFAs are suitable for use in a hydro-processing step to produce products suitable for use as transportation fuels, and the generated glycerol can be used in a hydrogenolysis step for producing propylene glycol.

### RELATED APPLICATIONS

This Patent Convention Treaty (PCT) International Application claims the benefit of priority under 35 U.S.C. § 119(e) of U.S. Provisional Application No. U.S. Ser. No. 63/256,449, Oct. 15, 2021. The aforementioned application is expressly incorporated herein by reference in its entirety and for all purposes. All publications, patents, patent applications cited herein are hereby expressly incorporated by reference for all purposes.

### TECHNICAL FIELD

This invention generally relates to biofuels and biorenewable feedstock processing. In alternative embodiments, provided are processes for the integration of hydrolysis of renewable glycerides such as mono- di- and tri-glycerides for the subsequent generation of paraffins (or a mixture of saturated hydrocarbons, or a mixture of alkanes containing around 80% to 90% linear chains (n-paraffin) with about 20 to 30 carbons in length) and propylene glycol from bio-feedstocks such as plant oils, animal oils, animal fats, and greases. In alternative embodiments, provided are processes for producing paraffins useful as transportation fuel and polyols from renewable feedstocks such as the glycerides and free fatty acids found in materials such as plant oils, animal oils, animal fats, and greases. In alternative embodiments, processes as provided herein comprise integrating a glyceride hydrolysis step to liberate the glycerol from the glyceride feed stock, whereby the resulting free fatty acids are subjected to a hydro-processing step to form paraffins and the glycerol is subjected to hydrogenolysis to form propylene glycol.

### BACKGROUND

As the demand for fuels such as aviation fuel and chemicals such as propylene glycol increase worldwide, there is increasing interest in sources other than petroleum crude oil for producing the fuel or chemical. One source is renewable feedstocks including, but not limited to, plant oils such as corn, jatropha, camelina, rapeseed, canola, soybean and algal oils, animal fats such as tallow, fish oils, and various waste streams such as yellow and brown greases and oily streams recovered from sewage sludge. The common feature of these feedstocks is that they are composed of mono- di- and tri-glycerides, and free fatty acids (FAAs). Most of the glycerides in the renewable feed stocks will be triglycerides, but some may be monoglycerides or diglycerides. The monoglycerides and diglycerides can be processed along with the triglycerides.

There are reports teaching the production of hydrocarbons from oils. For example, U.S. Pat. No. 4,300,009 teaches the use of crystalline aluminosilicate zeolites to convert plant oils (for example, corn oil) to hydrocarbons (for example, gasoline), and chemicals (fore example, para-xylene). U.S. Pat. No. 4,992,605 teaches the production of hydrocarbon products in the diesel boiling range by hydroprocessing vegetable oils such as canola or sunflower oil. US patent application publication no. 2004/0230085 A1 teaches a process for treating a hydrocarbon component of biological origin by hydrodeoxygenation followed by isomerization. U.S. Pat. No. 8,686,198 teaches an integrated hydrolysis/hydroprocessing process for converting feedstocks containing renewable glycerides to paraffins and polyols.

However, existing processes for converting fats and oils to biodiesel and glycerol have been criticized because of the quality of the diesel produced and the oversupply of glycerol. In addition, current processes for producing transportation fuel from renewable feedstocks have been criticized because of concerns over the high investment cost and operating costs. Thus, there remains a need for a process that maximizes the efficiency of the existing renewable diesel hydro-processing assets in the market and that allows for full utilization of a renewable glyceride source material.

### SUMMARY

In alternative embodiments, provided are improved and integrated processes for producing paraffins and propylene glycol from renewable feedstocks comprising first subjecting renewable glycerides to a hydrolysis process step to generate free fatty acids (FFAs) and glycerol. The generated FFAs are suitable for use in a hydro-processing step to produce products suitable for use as transportation fuels, and the generated glycerol can be used in a hydrogenolysis step for producing propylene glycol.

In alternative embodiments, provided are processes, including industrial processes and integrated processes, for producing paraffins (such as, for example, a C8 to C18 linear alkane and/or a branched alkane) and polyols (such as, for example: 1,2 propanediol (1,2 PDO); 1,3 PDO; ethylene glycol (or ethane-1,2-diol); 1,2 butanediol (BDO); 2,3 BDO and/or 1,4 BDO) from a glyceride-comprising renewable feedstock comprising:
(a) reacting the glyceride-comprising renewable feedstock with water in a first reaction zone to produce an effluent stream comprising a plurality of free fatty acids (FFAs), glycerol, and water, wherein optionally the amount of water in the initial reaction mixture (in the first reaction zone) is: between about 0.025% water to about 20% water, between about 0.05% water to about 10% water, between about 0.5% to 5% water or between about 0.25% to 1% water,
wherein optionally the reacting comprises reaction conditions comprising: a temperature in the range of between about 150° C. to 250° C. or 100° C. to 300° C., pressure at between about 300 psi to 500 psi (1 MPa is 145.038 psi (pound force per square inch)), and/or a reaction time of between about 0.1 hours (hrs) to 5 hrs, or between about 30 minutes to about 3 hours;
(b) separating or substantially separating the free fatty acids from the glycerol and water to produce a free fatty acid (FFA) stream substantially free of glycerol, water, and metals (wherein optionally the free fatty acid (FFA) stream is between about 90% to 99.9% free of glycerol, water, and metals), and a glycerol and water stream substantially free of FFAs (wherein optionally the glycerol and water stream is between about 90% to 99.9% free of FFAs);
(c) reacting the free fatty acid stream substantially free of glycerol, water, and metals of step (b) with hydrogen from a hydrogen source in a second reaction zone in the presence of a hydro-processing catalyst under hydro-processing conditions, thereby hydro-deoxygenating the free fatty acid stream substantially free of glycerol, water, and metals to produce a reaction product comprising n-paraffins, or C8 to C18 linear alkane paraffins and/or branched alkane paraffins, and water,
wherein optionally the hydro-processing conditions comprise: a temperature in the range of between about 200° C. to 500° C., or between about 280° C. to 450° C.; and/or a pressure of between about 1 to 5 MPa (megapascal) (1 MPa is 145.038 psi);
(d) recovering, isolating, or substantially isolating the n-paraffin reaction product, wherein optionally the substantially isolated n-paraffin reaction product is between about 80% to 99%, or is between about 90% to 99.9%, pure n-paraffin reaction product;
(e) reacting the stream comprising glycerol and water that is substantially free of free fatty acids with hydrogen from the hydrogen source in a third reaction zone under hydrogenation conditions to produce a reaction product comprising polyols,
and optionally the glycerol (or optionally, substantially purified glycerol), or the glycerol in the stream, is mixed with water, and with hydrogen it is processed to a gas phase and run over a copper catalyst material to become (or generate) primarily propylene glycol, and the conditions comprise a temperature of less than about 400° C. or 350° C., a hydrogen partial pressure of greater than about 3450 kPa or 3000 kPa, an H₂ to organic oxygen ratio greater than about 3, 4, or 5, and in the presence of a base metal catalyst, wherein optionally the base metal catalyst comprises sulfided nickel-molybdenum or cobalt-molybdenum on gamma alumina,
and optionally substantially purifying the glycerol comprises processes comprising absorbing free fatty acids (FFAs) on or using, for example, ion exchange or an absorbent, or adding CaO or equivalents to turn any residual FFA to insoluble soap, and them removing the soap; or alternatively, stripping (for example, steam stripping) all the glycerol away from the FFA or soaped FFA to get a clean (or substantially purified) glycerol, then adding water back in,
wherein optionally the amount of water is: between about 0.025% water to about 20% water, between about 0.05% water to about 10% water, between about 0.5% to 5% water or between about 0.25% to 1% water;
wherein optionally the hydrogenation conditions comprise: a temperature in the range of between about 190° C. to 350° C., or between about 210° C. to 220° C., or between about 100° C. to 500° C., or between about 200° C. to 300° C., or between about 280° C. to 450° C.; and/or a pressure of between about 0.5 to about 2 MPa or between about 1 to about 5 MPa, or about 0.25 to about 7 MPa, or about 2 to about 4 MPa, or between about 0.1 to 1.5 MPa (megapascal); and
(f) recovering, removing or substantially isolating the polyol reaction product, wherein optionally the polyol reaction product comprises: 1,2 propanediol (1,2 PDO); 1,3 PDO; ethylene glycol (or ethane-1,2-diol); 1,2 butanediol (BDO); 2,3 BDO and/or 1,4 BDO.

In alternative embodiments, in alternative embodiments of processes, including integrated processes, as provided herein:
the processes further comprise contacting at least a portion of the n-paraffin reaction product with an isomerization catalyst under isomerization conditions to isomerize at least a portion of (or at least about 1%, 5%, or 10% of, or between 0.5% to 15% of, or between about 1% to 50% of) the n-paraffins to isoparaffins,
and optionally the isomerization conditions comprise between about 350° C. to 450° C. or 300° C. to 500° C., and/or a pressure of between about 3 MPa to 5 MPa, or between about 2 MPa to 7 MPa,
and optionally isomerization catalyst comprises or is fabricated as or from a molecular sieve and/or a metal selected from, or comprising: an element from Group VIII of the Periodic Table (for example, iron (Fe), ruthenium (Ru), osmium (Os) and/or hassium (Hs)) and/or a carrier; optionally the isomerization catalyst comprises: a medium-pore silico-alumino-phosphate molecular sieve with tunable acidity having regularly alternating ALO₄, PO₄, and SiO₄ tetrahedra, and 10-ring channels that are unidirectional and non-intersecting with elliptical pore apertures ranging from 0.39 nm to 0.63 nm, e.g., SAPO-11™; or SAPO-41™; or an orthorhombic high silica zeolite having a framework of 5-, 6- and 10-rings, containing ferrierite sheets, and having a channel system that is linear unidirectional and one-dimensional (noninterconnecting) with 10-membered ring openings which can be 5.5×4.5 Å, or ZSM-22™, or a high-silica zeolite molecular sieve having a framework with pores defined by parallel channels that have cross-sectional dimensions of about 4.5 angstrom by about 5.2 angstrom, e.g., ZSM-23™; or ferrierite and/or Pt, Pd or Ni and Al2O₃ or SiO₂. Typical isomerization catalysts are, for example, Pt/SAPO-11/Al2O₃, Pt/ZSM-22/Al2O₃, Pt/ZSM-23/Al2O₃ and Pt/SAPO-11/SiO₂; most of these catalysts require the presence of hydrogen to reduce the catalyst deactivation;
and in alternative embodiments, separating the free fatty acids from the glycerol and water comprises at least one process (or two or more processes) comprising: hot depressurization, cyclonic vapor liquid separation, liquid-liquid separation, multi-effect evaporation, steam distillation, vacuum distillation, reactive distillation, liquid extraction, adsorption, filtration, membrane separation, absorption and/or ion exchange;
and in alternative embodiments, separating or substantially separating the free fatty acids from the glycerol and water comprises: separating or substantially separating the effluent stream into a free fatty acid-enriched stream (wherein optionally a free fatty acid (FFA)-enriched stream has more FFA than the effluent stream from which is was derived, or has between about 1% to 50% more FFA than the effluent stream from which it was derived) and a glycerol-comprising stream; and, purifying or substantially purifying the free fatty acid (FFA)-enriched stream to form the free fatty acid stream substantially free of glycerol, water, and metals;
and in alternative embodiments, the processes further comprise purifying or substantially purifying the glycerol containing stream to form the glycerol and water stream substantially free of free fatty acids;
and in alternative embodiments, the free fatty acid stream substantially free of glycerol, water, and metals has less than about 5 wt % glycerol, less than about 5 wt % water, and less than about 20 ppm (wt) metals, or, has less than about 10 wt % glycerol, less than about 10 wt % water, and less than about 40 ppm (wt) metals, or, has less than about 5 wt % glycerol, less than about 5 wt % water, and less than about 10 ppm (wt) metals;
reacting the glyceride-comprising renewable feedstock with water takes place in the presence of a hydrolysis catalyst, wherein optionally the hydrolysis catalyst comprises a mineral acid (such as, for example, hydrochloric acid, sulfuric acid, trifluoroacetic acid, formic acid, nitric acid), or hot compressed water as in a so-called “fat-splitting” procedure;
reacting the glyceride-comprising renewable feedstock with water is performed at sub-critical conditions, and optionally the subcritical conditions comprise pressure of less than about 373° C. and 220 bars (1 MPa is equivalent to 10 bar);
reacting the glyceride-comprising renewable feedstock with water takes place at a pressure of less than about 5516 kPa(g) and a temperature of less than about 300° C.;
the glyceride-comprising renewable feedstock comprises algae or a stream derived from algae;
the processes further comprise contacting the glyceride-comprising renewable feedstock with one or more of an acid, a base, an extractive material, an adsorptive material or any combination thereof to remove or substantially remove contaminants from the glyceride-comprising renewable feedstock before reacting the glyceride-comprising renewable feedstock with the water;
the hydroprocessing conditions comprise a temperature of less than about 400° C. or 350° C., a hydrogen partial pressure of greater than about 3450 kPa or 3000 kPa, an H₂ to organic oxygen ratio greater than about 3, 4, or 5, and in the presence of a base metal catalyst, and optionally the hydroprocessing conditions comprise a temperature of less than about 400° C. or 350° C., a hydrogen partial pressure of greater than about 3450 kPa or 3000 kPa, an H₂ to organic oxygen ratio greater than about 3, 4, or 5, and in the presence of a base metal catalyst, wherein optionally the base metal catalyst comprises sulfided nickel-molybdenum or cobalt-molybdenum on gamma alumina, and suitable active metals for this type of catalyst include molybdenum, chromium, tungsten, copper, and a metal from Group VIB (for example, chromium (Cr), molybdenunm (Mo) and/or tungsten (W)), VIIB (for example, fluorine (F), chlorine (Cl), bromine (Br), iodine (I), and/or astatine (At)), and IB (for example, copper, silver and/or gold) of the periodic table, as fully a reduced metal and/or a metal sulfide—at least about 70 wt %, 75 wt %, 80 wt %, 85 wt % or 90 wt % of the total lipids in the glyceride-comprising renewable feedstock are converted to free fatty acids in the reaction with water;
reacting the glyceride-comprising renewable feedstock with water, a ratio of water to total lipids in the first reaction zone is greater than a stoichiometric minimum required for complete hydrolysis;
at least about 80 wt %, 85 wt % or 90 wt % (or, at least about 50%, or about 60%, or about 70%, or about 75%) of oxygen in the free fatty acid stream substantially free of glycerol, water, and metals is converted to water and less than about 10% (or less than about 15%, or 5% or 1%) of carbon in the free fatty acid stream substantially free of glycerol, water, and metals is reacted to form CO or CO₂ in the second reaction zone;
at least about 75 wt %, 80%, 85 wt % or 90 wt % (or, at least about 50%, or about 60%, or about 70%, or about 75%) of the n-paraffins in the reaction product have an even number of carbon atoms;
reacting the free fatty acid stream substantially free of glycerol, water, and metals with hydrogen comprises selective catalytic hydrodeoxygenation (HDO) using an HDO catalyst (wherein optionally an HDO catalyst comprises sulfided nickel-molybdenum or cobalt-molybdenum on gamma alumina);
the third reaction zone the gas phase reaction mixture includes hydrogen at a partial pressure of hydrogen in a range from between about 0.01 and about 25 bars of hydrogen, or between about 0.1 and about 15 bars of hydrogen, or between about 1 and about 10 bars of hydrogen;
in the third reaction zone the gas phase mixture of glycerol, hydrogen and water is contacted with a heterogeneous catalyst that comprises at least one element from Groups I (for example, alkali metals such as hydrogen (H), lithium (Li), sodium (Na), potassium (K), rubidium (Rb), cesium (Cs), and/or francium (Fr)) or VIII (for example, iron (Fe), ruthenium (Ru), osmium (Os) and/or hassium (Hs)) of the Periodic Table, or ruthenium, copper, chromite, nickel and/or combinations thereof;
the third reaction zone the gas phase mixture of glycerol, hydrogen and water comprises essentially or substantially no liquid and has a partial pressure of glycerol between about 0.01 and about 0.5 bars (or between about 0.1 and about 1 bars) of glycerol; and establishing a temperature in a range from about 80° C., 85° C. or 90° C. to about 300° C. (or about 100° C. to about 250° C.) to facilitate a reaction;
the third reaction zone the gas phase mixture of glycerol, hydrogen and water comprises between about 0.05% to 5% water, or between about 1% to 2% water, or between about 1.0% to 50% water, or about 1%, 1.5%, 2%, 3%, 4%, 5%, 10% 15%, 20% or 25% or more water,
and optionally further comprising heating the reaction mixture in the third reaction zone to a temperature ranging from between about 150° C. to 250° C., or 125° C. to 300° C., over a reaction time interval ranging from between about 0.1 seconds to 3,600 seconds (or one hour) or at a time interval of between about 1 min to 90 minutes, at a pressure ranging from between about 1 and 25 bar, or from between about 1 and 25 bar, or alternatively, reaction conditions comprise between about 200° C. to 220° C. for about 2 to 5 seconds or 1 to 10 seconds, at about 5 bar or 3 to 7 bar;
the glyceride-comprising renewable feedstock is comprised of or derived from a microbial (for example, a bacterial, a fungal), an algal (for example, cyanobacteria (Cyanophyceae), green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) and ‘pico-plankton’ (Prasinophyceae and Eustigmatophyceae)), a plant and/or an animal material or source or any combination thereof, and optionally the microbial, the plant and/or an animal material or source comprises a microbial, a plant and/or an animal oil, optionally a corn, jatropha, camelina, rapeseed, canola, soybean and/or algal oil, or canola oil, corn oil, soy oils, rapeseed oil, soybean oil, colza oil, tall oil, sunflower oil, hempseed oil, olive oil, linseed oil, coconut oil, castor oil, peanut oil, pennycress oil, palm oil, carinata oil, jojoba oil, mustard oil, cottonseed oil, jatropha oil, tallow, yellow and brown greases, lard, train oil, fats in milk, fish oil, algal oil, sewage sludge, or an animal fat, wherein optionally the animal fat comprises or is derived from a tallow, a fish oil, a waste streams, a yellow and/or a brown grease, an oily stream recovered from sewage sludge, or any combination thereof, and/or
the glyceride in the glyceride-comprising renewable feedstock comprises mono-glycerides (such as for example, acylglycerols or monoacylglycerols, such as for example, monolaurin, glyceryl hydroxystearate or glycerol monostearate), diglycerides or diacylglycerol (DAG) (such as for example, 1,2-diacylglycerols and/or 1,3-diacylglycerols), and/or tri-glycerides or triacylglycerol (TAG), such as for example palmitic acid, oleic acid and/or alpha-linolenic acid.

In alternative embodiments, provided is an industrial plant for integrated hydrolysis/hydroprocessing processing comprising components for practicing a method as provided herein, for example, an industrial plant comprising components as described in FIG. 1 and/or FIG. 2.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

FIG. 1 is a schematic of one exemplary embodiment of an integrated hydrolysis/hydro-processing process, as discussed in further detail, below.

FIG. 2 is a schematic of one exemplary embodiment of a separation step, as discussed in further detail, below.

Like reference symbols in the various drawings indicate like elements.

### DETAILED DESCRIPTION

In alternative embodiments, provided are processes for producing a paraffin stream having boiling points in the diesel, naphtha, and aviation fuel range from renewable feedstocks, for example, biological feedstocks, originating from plants or animals other than petroleum feedstocks. By integrating hydrolysis and hydro-processing, the same fats and oils used or generated by known biodiesel processes can be converted to paraffins suitable for use as transportation fuels.

In alternative embodiments, processes as provided herein produce a more valuable co-product: polyol instead of glycerol. By integrating the hydrolysis and hydro-processing steps, common equipment can be used to minimize investment cost, raw material consumption, and energy requirements. In alternative embodiments, integrated processes as provided herein also allow for the use of renewable feedstocks containing high levels of metals without damaging the hydro-processing catalysts. In one embodiment, the process uses less hydrogen than the previous process.

In alternative embodiments, processes as provided herein produce a paraffin comprising a C8 to C18 linear alkane and/or a branched alkane, and optionally processes as provided herein produce a polyol comprising 1,2 propanediol (1,2 PDO); 1,3 PDO; ethylene glycol (or ethane-1,2-diol); 1,2 butanediol (BDO); 2,3 BDO and/or 1,4 BDO.

In alternative embodiments, integrated processes as provided herein can produce green diesel from natural oils and fats. In alternative embodiments, the process involved deoxygenating renewable feedstocks with carbon chain lengths in the diesel range to produce n-paraffins with both the same number of carbons as the fatty acid chain, or one carbon less if the oxygen was removed by decarboxylation or decarbonylation.

In an optional second stage of the process, a portion of the n-paraffins is selectively isomerized to improve the cold properties of the resulting diesel. The process can use a significant amount of hydrogen to support the chemistry of converting the triglycerides to diesel fuel and propane.

Provided herein are alternative methods for producing renewable hydrocarbons from lower cost or more sustainable feedstocks, which is desirable both from an economic standpoint, for example, having lower raw material costs and more valuable co-products, and from an environmental standpoint, for example, less greenhouse gas emissions. In addition, in alternative embodiments, reduced hydrogen consumption is achieved.

In alternative embodiments, at least a portion of the metals are removed from the feedstock before the feed reaches the hydrodeoxygenation (HDO) catalyst (wherein optionally an HDO catalyst comprises sulfided nickel-molybdenum or cobalt-molybdenum on gamma alumina) because, for example, renewable feedstocks may contain a variety of impurities. For example, the renewable feedstocks may contain contaminants comprising alkali metals, e.g. sodium and potassium, phosphorous, gums, and water. In alternative embodiments the renewable feedstocks comprise from about 10 ppm (wt) to over 5000 ppm (wt) of total metals. By “total metals” we mean the combined amount of Si, Fe, Al, K, Na, Mg, Cu, Ca, Ti, Mn, Zn and P in the feedstock. These metals can be deleterious to the hydro-deoxygenation catalyst, a problem which is solved by first removing at least a portion of the metals before the feedstock is contacted with a hydro-deoxygenation catalyst. In alternative embodiments, at least a portion of the metals are removed from the feedstock using a process comprising: filtration, settling and/or liquid solid contacting.

In alternative embodiments, a hydrolysis process is integrated with the hydroprocessing step to produce a paraffin product suitable for use as a transportation fuel. Integration reduces overall hydrogen consumption, process energy input and freshwater consumption and allows the use of common equipment. Taken together, these exemplary improvements as implemented by processes as provided herein translate into lower operating costs and/or lower capital investment per unit of paraffin product.

In alternative embodiments, in the hydrolysis step, water is reacted with the glyceride-containing feedstock to produce the corresponding FFA molecules and glycerol by hydrolysis chemistry. The hydrolysis can be thermal or catalytic. The reaction products are separated into a free fatty acid stream substantially free of glycerol, water, and metals, and a glycerol containing stream.

In alternative embodiments, after separation, the FFAs are processed as discussed above to obtain a paraffin-rich product (for example, rich in isoparaffin or n-paraffin, including kerosene), or a paraffinic diesel fuel having a high cetane rating or number (cetane number denotes the ignition delay time (the start of the injection of diesel fuel to the onset of the auto-ignition)), which can be suitable for use as a transportation fuel, and the glycerol is converted into polyol. In one embodiment, the net result is a shift from converting the triglycerides to diesel and propane to converting the triglycerides to diesel and propylene glycol while minimizing the overall hydrogen consumption.

In alternative embodiments, by “glyceride-containing feedstock,” we mean a stream comprising at least about 5 wt % total glycerides by dry weight, or at least about 10 wt %, or at least about 20 wt %, or at least about 30 wt %, or at least about 40 wt %, or at least about 50 wt %.

In alternative embodiments, by “free fatty acid stream substantially free of glycerol, water, and metals” we mean that the stream contains less than about 10 wt % glycerol, or less than about 5 wt % glycerol, or less than about 3 wt % glycerol, or less than about 1 wt % glycerol, or less than about 0.75 wt %, or less than 0.50 wt %. In alternative embodiments, there is less than about 10 wt % water, or less than about 5 wt % water, or less than about 3 wt %, or less than about 1 wt %.

In alternative embodiments, the stream has less than about 20 ppm (wt) total metals, or less than about 15 ppm (wt), or less than 10 ppm (wt).

The higher amounts of glycerol and water in the free fatty acid stream substantially free of glycerol water and metals can be less desirable because the economics are not as favorable; however, in certain situations, they may be acceptable. In alternative embodiments, by “glycerol stream substantially free of free fatty acids,” we mean a stream comprising less than about 10 wt %, or 5 wt %, or 3 wt %, of the total amount of fatty acids present in the glyceride-containing feedstock. In alternative embodiments, by “glycerol containing stream,” we mean a stream comprising at least about 40% (wt), 30% (wt) or 20% (wt) glycerol; it may contain water (for example, between about 0.1% and 5% water), and possibly some (for example, between about 0.1% and 5%) FFAs.

In alternative embodiments, the hydrolysis step is performed at sub-critical conditions. In alternative embodiments, conditions for the hydrolysis step are less than about 300° C. and less than about 5516 kPa (g) (800 psig), or less than about 250° C. and less than about 3447 kPa (g) (500 psig), or less than about 150° C. and less than about 2068 kPa (g) (300 psig). In alternative embodiments, while critical and near critical conditions (supercritical water is defined as greater than about 374° C. and about 22,063 kPa (g) (3200 psig)) can be used, in alternative embodiments use of critical and near critical conditions are less desirable because as conditions approach critical conditions, the water mixture becomes highly corrosive, and the metallurgical requirements become cost prohibitive.

In alternative embodiments, the ratio of water to total lipids in the hydrolysis step is typically greater than the stoichiometric minimum required for complete hydrolysis. In some embodiments, a hydrolysis catalyst is needed in order to achieve some desirable operating conditions. Suitable hydrolysis catalysts include, but are not limited to, acidic catalysts. The hydrolysis catalyst can be a solid or a liquid. Suitable liquid hydrolysis catalysts include, but are not limited to, ionic liquids. Suitable solid hydrolysis catalysts include, but are not limited to, solid acid catalysts. The solid hydrolysis catalyst can be supported on a material that is stable at hydrolysis conditions. Examples of catalyst support materials which are stable over part of the range of operating conditions that can be used in exemplary processes as provided herein include, but are not limited to, amorphous carbon, activated carbon, titanium oxide and zirconium oxide.

In alternative embodiments, the hydrolysis step is preceded by a feed pretreatment step to remove contaminants such as metals, nitrogen, sulfur and other inorganic species. Feed pretreatment is discussed further below. The pretreatment can be designed and operated to retain at least about 80%, or at least about 90%, or between about 70% and 95%, of the total lipid and free fatty acids present in the feedstock in the hydrolysis reactor feed. In alternative embodiments, by “total lipids,” we mean any of a group of organic compounds, including the fats, oils, waxes, sterols, and glycerides, that are insoluble in water but soluble in organic solvents, and are oily to the touch.

In alternative embodiments, although conventional processes utilize a dry feedstock, exemplary processes as provided herein do not necessarily require the renewable feedstock to be dry, or substantially dry, or partially dehydrated. In alternative embodiments, the feedstock can contain more than about 1%, 5% or 10% moisture, or water, or between about 0.5% and 20% by volume water, and in alternative exemplary processes, the feedstock moisture content can be as much as five (or between about 2 and 10) times the total lipid plus free acid concentration.

In alternative embodiments, feedstocks used in processes as provided herein include or comprise all or any renewable source of lipids and/or free fatty acids. In alternative embodiments, special emphasis is placed on low-cost waste-derived feedstocks, such as brown grease, yellow grease, inedible tallow, used cooking oils, and mixtures containing these materials. The feedstock total lipid to fatty acid ratio can be from between about 0.05 to 0.995, or between about 0.01 and 0.90, on a molar basis depending on feedstock source.

Other feedstocks that can be used in exemplary processes as provided herein include crude and refined vegetable oils with special emphasis on lower cost crude vegetable oils unsuitable for direct use as feedstock for transesterification or hydro-conversion processes due to the presence of alkali metals, gums and phospholipids. In alternative embodiments, a desirable feedstock is one containing or comprising algal lipids, and in alternative embodiments, an algal feedstock used in exemplary processes as provided herein are enriched in total lipids plus free fatty acids by pretreatment to remove proteins and cellulose (for example, by pretreatment with mild acid hydrolysis). Suitable feedstocks are discussed in more detail below.

In alternative embodiments, the hydrolysis step is operated to convert at least about 70%, about 75%, about 80%, about 85%, at least about 90%, or at least about 95% to 99% of the total lipids present in the feedstock (including polar lipids) to free fatty acids. In alternative embodiments, at least about 5%, about 10%, about 15% or about 20% of the polar lipids are converted to FFA by hydrolysis.

In alternative embodiments, the hydrolysis step is continuous; and/or, in alternative embodiments, the hydrolysis step operates at steady state conditions, under constant temperature, pressure, and flow.

In one embodiment, the ratio of water to total lipids is greater than the stoichiometric requirement, or in excess of about three (3) times the stoichiometric requirement needed for hydrolysis.

In alternative embodiments, the recovery of free fatty acid (FFA) from the hydrolysis reactor involves a series of steps including, but not limited to, one or more of the following: hot depressurization, cyclonic vapor-liquid separators, liquid-liquid separators, multi-effect evaporators, steam distillation, vacuum distillation, reactive distillation, liquid extraction, adsorption, filtration, membrane separation, absorption, and/or ion exchange; and in alternative embodiments, this recovery produces a FFA stream of adequate purity to be directly fed to a fixed bed catalytic hydro-deoxygenation reactor for conversion of the FFA to hydrocarbon fuel. In alternative embodiments, the stream entering the hydrodeoxygenation (HDO) reactor includes less than about 20 ppm (wt) total metals (or between about 5 to 35 ppm (wt) total metals), less than about 5% by weight (or between about 1% to 15% by weight) total glycerol, and less than about 5% by weight (or between about 1% to 15% by weight) total water.

In alternative embodiments, at least about 90% (or between about 70% and 99%) of the FFA present in the hydrolysis reactor is recovered in the FFA recovery section of the process.

In alternative embodiments, the deoxygenation zone is catalytic or thermal, and/or selective or non-selective. In one embodiment, selective catalytic hydrodeoxygenation (HDO) can be used to maximize the yield of paraffins suitable for use as transportation fuels.

In alternative embodiments, selective catalytic hydrodeoxygenation (HDO) provides several benefits. For example, less than about 10% of the FFA carbon is lost to CO/CO₂ by decarboxylation and decarbonylation reactions. The H₂ partial pressure at the hydrodeoxygenation reactor outlet is greater than about 3447 kPa (500 psia), or more than about 5516 kPa (800 psia). The hydrogen supplied to the reactor is generally at least three times the stoichiometric requirement for 100% conversion of feedstock organic oxygen to water. Thus, in alternative embodiments, more than about 80% of the oxygen in the free fatty acid stream substantially free of glycerol, water, and metals is converted to water. The resultant hydrocarbon can be distillate (diesel plus kerosene) with an aromatic content of less than about 1% and a paraffin content of more than about 90%.

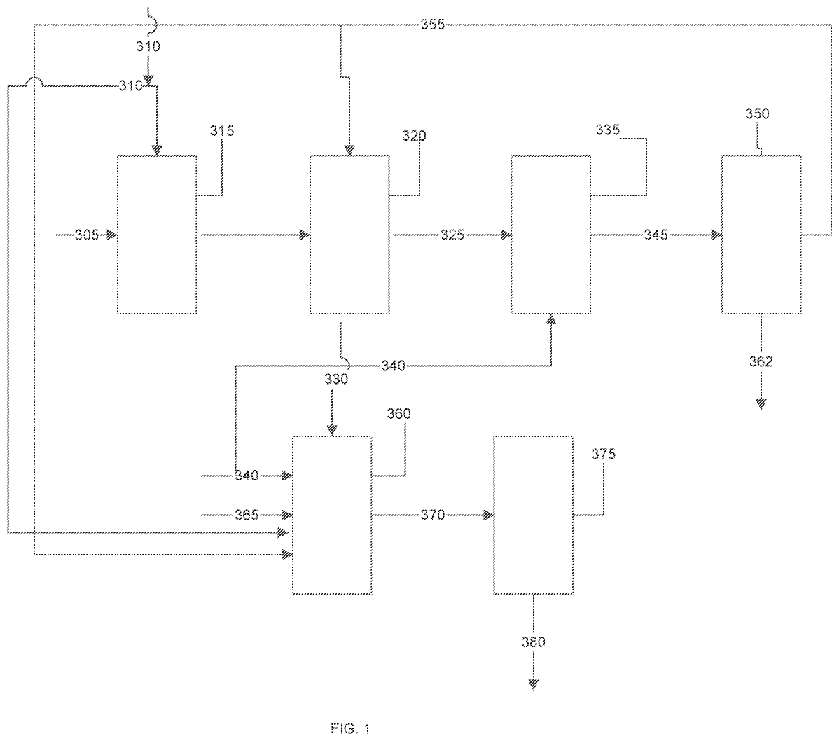

In alternative embodiments, the hydrodeoxygenation (HDO) zone effluent is routed to a selective isomerization zone for improving distillate cold flow properties while minimizing cracking to naphtha and lighter hydrocarbons, if desired. In alternative embodiments, water produced in the hydrodeoxygenation (HDO) zone is recycled as at least a portion of the make-up water to the hydrolysis step (see for example FIG. 1).

In some embodiments, at least a portion of the hydrogen supplied to the hydrogenation reactor is excess hydrogen recovered from the reactor product and recycled to the reactor to achieve the hydrogen partial pressure targets discussed above.

In alternative embodiments, recovery of glycerol from the hydrolysis reactor comprises a series of steps such as those described above with respect to the FFA stream to produce a glycerol stream of adequate purity for the selective hydrogenation step. In alternative embodiments, the selective hydrogenation step uses a noble metal catalyst. In alternative embodiments, the process operates at a temperature in the range of between about 150° C. to about 250° C., a pressure in the range of between about 3447 kPa (g) (500 psig) to about 13790 kPa (g) (2000 psig), and a weight hourly space velocity (WHSV) of between about 0.2 to about 2 hr⁻¹. In alternative embodiments, small amounts (which can be in the range of between about 1 wt % to about 3 wt %) of alkali (NaOH) are injected into the reaction zone to improve the activity and/or the selectivity.

In one embodiment as shown in FIG. 1, a pretreated renewable glyceride-rich feedstock 305 and water 310 enter a hydrolysis zone 315. The glycerides are hydrolyzed to FFAs and glycerol. The FFAs and glycerol hydrolysis products enter a separation zone 320. The FFAs and glycerol are separated (or substantially separated) into a free fatty acid stream substantially free of glycerol, water, and metals 325 and a glycerol containing stream 330. The separation zone 320 is described in more detail below. The free fatty acid stream substantially free of glycerol, water, and metals 325 is sent to the hydroprocessing zone 335 where the FFAs are reacted with hydrogen from hydrogen source 340 to produce a paraffin product useful as a transportation fuel 345 which is sent to recovery zone 350 where the n-paraffin product 362 is recovered.

In alternative embodiments, optionally, recycle water 355 from the separation zone 320 and/or the paraffin recovery zone 350 can be combined with make-up water to provide at least a portion of the water stream 310 for the hydrolysis zone 315. The glycerol containing stream 330 and water 310 and or water 355 is sent to selective hydrogenation zone 360, where it is the glycerol and water are mixed and then reacted with hydrogen from hydrogen source 340 in the presence of a hydrogenation catalyst 365. The polyol product 370 is then recovered in polyol recovery zone 375 as polyol product 380.

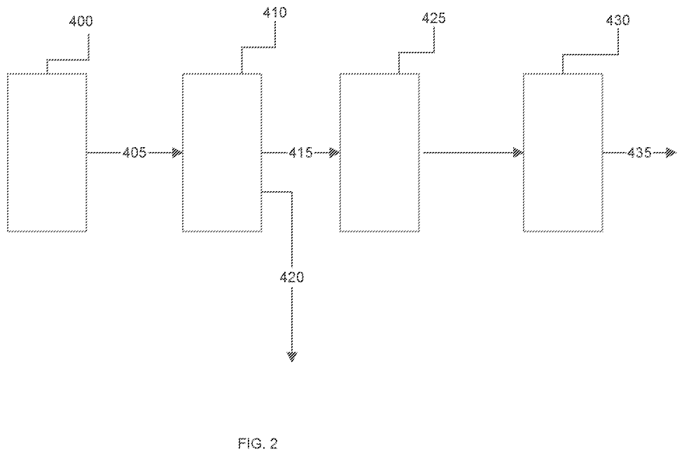

One embodiment of a process for separating the hydrolysis products into the free fatty acid stream substantially free of glycerol, water, and metals and the glycerol-containing stream is illustrated in FIG. 2. The effluent 405 from the hydrolysis zone 400 enters extraction column 410. The extraction column 410 separates the product into a free fatty acid enriched stream 415 and a glycerol-containing stream 420. The free fatty acid enriched stream 415 is optionally sent to one or more purification devices 425, 430 to remove water, glycerol, and metals producing the free fatty acid stream substantially free of glycerol, water, and metals 435. The free fatty acid stream substantially free of glycerol, water, and metals 435 is sent to the hydroprocessing zone where it is reacted with hydrogen in the presence of a catalyst to hydrodeoxygenate it, producing n-paraffins and water.

In alternative embodiments, similar processes and equipment are used to separate and purify (or substantially separate and/or purify) the glycerol containing stream to the glycerol stream substantially free of free fatty acids.

In alternative embodiments, the operating conditions and catalyst types are adjusted for the hydrolysis and hydrodeoxygenation processes to control the amount of organic oxygen in the feed which is converted to CO₂ instead of water, if desired.

In alternative embodiments, suitable renewable feedstocks used in processes as provided herein include those originating from plants or animals, including feedstocks known as renewable fats and oils. In alternative embodiments, the term renewable feedstock is meant to include a feedstock other than one obtained from petroleum crude oil. In alternative embodiments, renewable feedstocks used include any of those which comprise glycerides and FFA. Most of the glycerides will be triglycerides, but monoglycerides and diglycerides may be present and processed as well. Examples of these feedstocks used in processes as provided herein include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, pennycress oil, palm oil, carinata oil, jojoba oil, mustard oil, cottonseed oil, jatropha oil, tallow, yellow and brown greases, lard, train oil, fats in milk, fish oil, algal oil, sewage sludge, and the like. Additional examples of renewable feedstocks used in processes as provided herein include non-edible vegetable oils from the group comprising Jatropha curcas (Ratanjoy, Wild Castor, Jangli Erandi), Madhuca indica (Mohuwa), Pongamia pinnata (Karanji Honge), and Azadiracta indicia (Neem).

Triglycerides and FFAs of the typical vegetable or animal fat used in processes as provided herein contain aliphatic hydrocarbon chains in their structure which have about 8 to about 24 carbon atoms, with a majority of the fats and oils containing high concentrations of fatty acids with 16 and 18 carbon atoms. Mixtures or co-feeds of renewable feedstocks and petroleum derived hydrocarbons may also be used as the feedstock used in processes as provided herein.

In alternative embodiment, other feedstock components are used, especially as a co-feed component in combination with the above listed feedstocks, and can include spent motor oils and industrial lubricants, used paraffin waxes, liquids derived from the gasification of coal, biomass, or natural gas followed by a downstream liquefaction step such as Fischer-Tropsch technology (for example, (2n+1) H₂+n CO→CnH₂n+2+n H₂O), liquids derived from depolymerization, thermal or chemical, of waste plastics such as polypropylene, high density polyethylene, and low density polyethylene; and other synthetic oils generated as byproducts from petrochemical and chemical processes. Mixtures of the above feedstocks may also be used as co-feed components. In some exemplary embodiments, an advantage of using a co-feed component is the transformation of what may have been considered to be a waste product from a petroleum based or other process into a valuable co-feed component.

Algae that can be used in processes as provided herein are one type of biomass that is of particular interest because they are one of the fastest growing plants on the planet, therefore offering one of the highest yields per unit area. Algae also do not need arable land, and can be grown with impaired water. Algae have been used as a feedstock to produce biofuel using various methods. Algae used in processes as provided herein can contain neutral lipids (triacylglycerols (TAGs)), glycolipids found in algal chloroplast membranes (for example, monogalactosyldiacylglycerols and digalactosyldiacylglycerols), and polar lipids of the algal plasma membranes, primarily phospholipids (for example, phosphatidylcholine). The glycolipids and other polar lipids represent a significant portion of the total lipids in the algae. Conventional production methods of making biofuel from algae use only a fraction (the neutral lipids) of the total available lipid material in the algae leaving a large percentage of “residual algal biomass” remaining after the TAG lipids have been extracted to form a neutral oil extract. Conventional methods are incapable of converting the glycolipids and other polar lipids in the algae into biofuel. These lipids cannot be extracted from algal biomass by conventional methods, and thus, cannot be recovered by downstream hydrolysis. Therefore, conventional methods produce only a small fraction of the energy that can potentially be obtained from the algae.

In alternative embodiments, processes as provided herein comprise making hydrocarbons from algal biomass, for example, as described in US Patent Publication No. 2011/0287503. In alternative embodiments, the algal biomass is formed from algae, and the term “algae” as used herein refers to any organisms with chlorophyll and a thallus not differentiated into roots, steams and leaves, and encompasses prokaryotic organisms such as any organism from the phylum Cyanobacteria (blue-green algae), including Melainabacteria and Oxyphotobacteria, and eukaryotic organisms (or any organism of the domain Eukaryota) that are photoautotrophic or photoauxotropic. In alternative embodiments, the term “algae” includes macroalgae (commonly known as seaweed) and microalgae. In alternative embodiments, algal biomass used in processes as provided herein comprise dried algae. In alternative embodiments, algae used in processes as provided herein include any species or strain in the following taxonomic groups: Bacillariophyceae (for example, diatoms), Charophyta (for example, freshwater green algae or stoneworts), CHLOROPHYTA (for example, green algae), Chrysophyta (for example, golden algae), Dinoflagellata (for example, dinoflagellates), Haeophyta or Phaeophyceae (for example, brown algae), Rhodophyta (for example, red algae), Cyanophyta, Prochlorophyta, glaucophytes or Glaucophyta, Cryptophyta, Prymnesiophyta, Xanthophyta, Rhaphidophyta, Phaeophyta, Nanochloropsis (for example, marine algae) and/or Eustigmatophytes (for example, algae). Examples of suitable algae used in processes as provided herein are also described in Section 5.2 of U.S. Publication No. 2010/0050502, which section is incorporated herein by reference.

Algal biomass may be provided by harvesting algae from a source such as a bioreactor, aquaculture pond, wastewater, lake, pond, river, sea, etc. The algae may be, for example, a naturally occurring species, a genetically selected strain, a genetically manipulated strain, a transgenic strain, a synthetic algae, or combinations thereof. Algae used in processes as provided herein can be grown as a dilute suspension.

The algae feedstock used in processes as provided herein can be either wet or dry. In alternative embodiments, using a wet feedstock is desirable because it is less expensive than dry feedstock. In one embodiment, to obtain a dry feedstock, the harvested algae can be dewatered, for example in a concentrator, and the concentrator can concentrate the algae producing the whole algal biomass. In alternative embodiments, the whole algal biomass comprises a concentrated algal paste of about 4% to about 12% solids by weight. In alternative embodiments, the concentrated algal paste is in the form of slurry with a paste-like consistency, able to be pumped from the concentrator into a reactor. Any one or more known methods for dewatering the algae in the concentrator can be used, including but not limited to, sedimentation, filtration, centrifugation, flocculation, froth floatation, and/or semipermeable membranes. In alternative embodiments, whole algal biomass used in processes as provided herein is also commercially available from sources such as algal cultivators, Solix Biofuels Inc., Fort Collins, Colo. (USA) and Cyanotech Corp., Hawaii (USA).

In alternative embodiments, the algae can undergo extraction, although this is not required. In alternative embodiments, to form the residual algal biomass, the whole algal biomass from concentrator is dried in a dryer by evaporation or the like to provide dried concentrated algal paste. In alternative embodiments, the neutral triacyglycerols (TAGs) are then extracted from the dried concentrated algal paste in a neutral lipid extractor by known lipid extraction methods using an organic solvent such as hexane or the like to produce a “neutral oil extract” (also referred to herein as “TAG oil”). In alternative embodiments, the TAG oil produced during the extracting step is withdrawn from the neutral lipid extractor. In alternative embodiments, a mixture of the organic solvent and TAG oil is provided to an input of an evaporator. In alternative embodiments, the organic solvent is evaporated in the evaporator leaving the TAG oil which can be sent to a downstream process.

In alternative embodiments, “residual algal biomass” is used as a feedstock, and it can refer to the dried bagasse remaining after the neutral lipids, for example, the triacylycerols (TAGs) are recovered as “TAG oil” by the solvent extraction. In alternative embodiments, residual algal biomass used as feedstock comprises polar lipids and glycolipids, residual protein and carbohydrates, and algal cell debris. In alternative embodiments, total mass of the bagasse after the solvent extraction of TAG oil is at least 70% of the total algal biomass.

In alternative embodiments, the algal biomass comprised of whole algal biomass from the concentrator, the TAG oil, or combinations thereof, can be introduced as feedstock to the first reactor. As discussed above, the renewable feedstock can be pretreated before entering the hydrolysis zone to remove contaminants. Suitable pretreatments include, but are not limited to, contacting the glyceride-containing renewable feedstock with one or more of a acid, a base, an extractive material, or an adsorptive material.

In alternative embodiments, one pretreatment step involves contacting the renewable feedstock with an ion-exchange resin in a pretreatment zone at pretreatment conditions. In one embodiment, the ion-exchange resin is an acidic ion exchange resin such as a macro-reticular polystyrene-based ion exchange resin with strongly acidic sulfonic group (such as for example, AMBERLYST 15™) and can be used as a bed in a reactor through which the feedstock is flowed, either upflow or downflow.

In alternative embodiments, contaminants are removed using a mild acid wash. This can be carried out by contacting the feedstock with an acid such as sulfuric, nitric or hydrochloric acid in a reactor. The acid and feedstock can be contacted either in a batch or continuous process. In alternative embodiments contacting is done with a dilute acid solution, optionally at ambient temperature and atmospheric pressure. In alternative embodiments, contacting is done in a continuous manner, and it also can be done in a counter current manner.

In alternative embodiments, metal contaminants are removed from the feedstock by using guard beds, which are well known in the art. These can include alumina guard beds either with or without demetallation catalysts such as nickel or cobalt. Filtration and solvent extraction techniques are other choices which may be employed.

In alternative embodiments, hydroprocessing such as that described in U.S. Pat. No. 7,638,040 or 8,038,869, or equivalent processes, are used as feedstock pretreatment techniques. For example, in alternative embodiments, the free fatty acid stream substantially free of glycerol, water, and metals from the separation zone flows to the hydroprocessing zone comprising one or more catalyst beds in one or more reactors. In the hydroprocessing zone, the free fatty acid stream substantially free of glycerol, water, and metals can be contacted with a hydrogenation or hydrotreating catalyst in the presence of hydrogen at hydrogenation conditions to hydrogenate the reactive components such as olefinic or unsaturated portions of the n-paraffinic chains. In alternative embodiments, hydrogenation and hydrotreating catalysts are any of those well known in the art such as nickel or nickel/molybdenum dispersed on a high surface area support. Other hydrogenation catalysts that can be used include one or more noble metal catalytic elements dispersed on a high surface area support. Non-limiting examples of noble metals include Pt and/or Pd dispersed on gamma-alumina, titanium oxide or activated carbon.

In alternative embodiments, hydrogenation conditions that are used in exemplary processes include a temperature of about between 40° C. to about 400° C. and a pressure of between about 689 kPa absolute (100 psia) to about 13,790 kPa absolute (2000 psia). Other operating conditions for the hydrogenation zone are well known in the art. For hydrodeoxygenation, the conditions can include a temperature of about 200° C. to about 400° C. and a pressure of about 4137 kPa absolute (600 psia) to about 8274 kPa absolute (1200 psia). In alternative embodiments, the hydrogen partial pressure is greater than about 3450 kPa absolute (500 psia). In alternative embodiments, the ratio of H₂ to organic oxygen is greater than about 5, or greater than about 7, or greater than about 10.

In alternative embodiments, suitable catalysts for hydrodeoxygenation include, but are not limited to, nickel or nickel/molybdenum containing catalysts.

In alternative embodiments, processes as provided herein also comprise decarboxylation, and decarbonylation in addition to hydrodeoxygenation of the feedstock to remove oxygen; and some of the catalysts enumerated above are also capable of, and used for, catalyzing decarboxylation, and decarbonylation in addition to hydrodeoxygenation of the feedstock to remove oxygen. Decarboxylation, decarbonylation, and hydrodeoxygenation are collectively referred to as deoxygenation reactions.

In alternative embodiments, decarboxylation and decarbonylation can be less desirable because of the loss of renewable carbon feedstock to CO and CO₂.

In alternative embodiments, decarboxylation conditions include a relatively low pressure of about 689 kPa (100 psia) to about 6895 kPa (1000 psia), a temperature of about 300° C. to about 450° C. and a liquid hourly space velocity of about 0.5 to about 10 hr¹.

Since hydrogenation is an exothermic reaction, as the feedstock flows through the catalyst bed, the temperature increases and decarboxylation and hydrodeoxygenation will begin to occur. Thus, in alternative embodiments, all the reactions (including deoxygenation and hydrogenation reactions) occur simultaneously in one reactor or in one bed.

Alternatively, in some embodiments the conditions can be controlled such that hydrogenation primarily occurs in one bed and deoxygenation (for example, decarboxylation and/or hydrodeoxygenation) occurs in a second bed. If only one bed is used, then hydrogenation occurs primarily at the front of the bed, while deoxygenation (for example, decarboxylation and/or hydrodeoxygenation) occurs mainly in the middle and bottom of the bed. Alternatively, in some embodiments desired hydrogenation can be carried out in one reactor, while decarboxylation, decarbonylation, and/or hydrodeoxygenation can be carried out in a separate reactor.

In alternative embodiments, the effluent from the deoxygenation zone is conducted to a hot high pressure hydrogen stripper. The reaction product from the deoxygenation reactions will comprise both a liquid portion and a gaseous portion. The liquid portion comprises a hydrocarbon fraction which is essentially all n-paraffins and having a large concentration of paraffins in the range of about 8 to about 18 carbon atoms. The gaseous portion comprises hydrogen, carbon dioxide, carbon monoxide, water vapor, propane, and perhaps sulfur components, such as hydrogen sulfide or phosphorous components such as phosphine, or nitrogen compounds such as ammonia. One purpose of the hot high pressure hydrogen stripper is selectively to separate at least a portion of the gaseous portion of the effluent from the liquid portion of the effluent. Thus, in alternative embodiments, processes comprise selective separation of at least a portion of the gaseous portion of the effluent from the liquid portion of the effluent using a hot high pressure hydrogen stripper.

Failure to remove the water, trace carbon monoxide, ammonia, and carbon dioxide from the effluent may result in poor catalyst performance in the isomerization zone. Thus, in alternative embodiments, water, carbon monoxide, carbon dioxide, and/or hydrogen sulfide are selectively stripped in the hot high pressure hydrogen stripper using hydrogen. The hydrogen used for the stripping may be dry, and free of carbon oxides.

In alternative embodiments, the temperature is controlled in a limited range to achieve the desired separation and the pressure may be maintained at approximately the same pressure as the two reaction zones to minimize both investment and operating costs. In alternative embodiments, the hot high pressure hydrogen stripper is operated at conditions ranging from a pressure of about 689 kPa absolute (100 psia) to about 13,790 kPa absolute (2000 psia), and a temperature of about 40° C. to about 350° C. In another embodiment, the hot high pressure hydrogen stripper may be operated at conditions ranging from a pressure of about 1379 kPa absolute (200 psia) to about 4826 kPa absolute (700 psia), or about 2413 kPa absolute (350 psia) to about 4882 kPa absolute (650 psia), and a temperature of about 50° C. to about 350° C. The hot high pressure hydrogen stripper may be operated at essentially the same pressure as the reaction zone. In alternative embodiments, by “essentially”, it is meant that the operating pressure of the hot high pressure hydrogen stripper is within about 1034 kPa absolute (150 psia) of the operating pressure of the reaction zone. For example, in one embodiment the hot high pressure hydrogen stripper separation zone is no more than about 1034 kPa absolute (150 psia) less than that of the reaction zone.

In alternative embodiments, the effluent from the deoxygenation reaction enters the hot high pressure stripper, and at least a portion of the gaseous components are carried with the hydrogen stripping gas and separated into an overhead stream. The remainder of the deoxygenation zone effluent stream is removed as hot high pressure hydrogen stripper bottoms and contains the liquid hydrocarbon fraction having components such as normal hydrocarbons having from about 8 to about 24 carbon atoms.

Different feedstocks will result in different distributions of paraffins. Thus, in alternative embodiments, a portion of this liquid hydrocarbon fraction in hot high pressure hydrogen stripper bottoms may be used as the hydrocarbon recycle described below. Hydrogen may be separated from process effluent(s) and recycled to the hydrogenation and deoxygenation zone, or the amount of hydrogen may be in only slight excess, about 5 to about 25%, of the hydrogen requirements of the hydrogenation and deoxygenation reactions and therefore not recycled. Another refinery unit, such as a hydrocracker, may be used as a source of hydrogen, which potentially eliminates the need for a recycle gas compressor.

In one embodiment, the desired amount of hydrogen is kept in solution at lower pressures by employing a large recycle of hydrocarbon to the deoxygenation reaction zone. In alternative embodiments, processes that employ use of hydrocarbon recycling are used to control the temperature in the reaction zones since the reactions are exothermic reactions.

However, the range of recycle to feedstock ratios is not always determined on temperature control requirements. In some cases, it is based upon hydrogen solubility requirements. Hydrogen has a greater solubility in the hydrocarbon product than it does in the feedstock. Thus, in alternative embodiments, by utilizing a large hydrocarbon recycle, the solubility of hydrogen in the combined liquid phase in the reaction zone is greatly increased, and higher pressures are not needed to increase the amount of hydrogen in solution. In one embodiment, the volume ratio of hydrocarbon recycle to feedstock is from about 2:1 to about 8:1, or about 2:1 to about 6:1. In another embodiment, the ratio is in the range of about 3:1 to about 6:1, and in yet another embodiment, the ratio is in the range of about 4:1 to about 5:1.

Although the hydrocarbon fraction separated in the hot high pressure hydrogen stripper is useful as a diesel boiling range fuel, it will have poor cold flow properties because it comprises essentially n-paraffins. Thus, in alternative embodiments, the hydrocarbon fraction is contacted with an isomerization catalyst under isomerization conditions to selectively isomerize at least a portion of the n-paraffins to branched paraffins to improve the cold flow properties. In alternative embodiments, the effluent of the isomerization zone is a branched-paraffin-rich stream. In alternative embodiments, by the term “rich” it is meant that the effluent stream has a greater concentration of branched paraffins than the stream entering the isomerization zone, and can comprises greater than about 15 mass-% or 20 15 mass-% branched paraffins. In alternative embodiments, the isomerization zone effluent contains greater than about 20, or greater than about 30, or greater than about 40, or greater than about 50, or greater than about 60, or greater than about 70, or greater than about 75, or greater than about 80, or greater than about 90 mass-% branched paraffins.

In alternative embodiments, isomerization is carried out in a separate bed of the same reaction zone, for example, same reactor described above for deoxygenation, or the isomerization can be carried out in a separate reactor. To be clear, an exemplary embodiment with a separate reactor for the isomerization reaction is described: the hydrogen stripped product of the deoxygenation reaction zone is contacted with hydrogen in the presence of an isomerization catalyst at isomerization conditions to isomerize the normal paraffins to branched paraffins. Only minimal branching is required, enough to overcome the cold-flow problems of the normal paraffins. Because attempting to obtain significant branching runs the risk of undesired cracking, the predominant isomerized product is a mono-branched hydrocarbon.

In alternative embodiments, the isomerization of the paraffinic product is accomplished in any manner known in the art or by using any suitable catalyst known in the art. One or more beds of catalyst may be used. In alternative embodiments, the isomerization is operated in a co-current mode of operation. In alternative embodiments, fixed bed, trickle bed down flow or fixed bed liquid filled up-flow modes are also suitable, or processes as described in US Pat application no. 2004/0230085 A1 are used.

In alternative embodiments, suitable catalysts used in processes as provided herein comprise a metal of Group VIII (IUPAC8-10) of the Periodic Table and a support material. Suitable Group VIII metals include platinum and palladium, each of which may be used alone or in combination. The support material may be amorphous or crystalline. Suitable support materials include, but are not limited to, amorphous alumina, titanium oxide, amorphous silica-alumina, ferrierite, ALPO-31, SAPO-11, SAPO-31, SAPO-37, SAPO-41, SM-3, MgAPSO-31, FU-9, NU-10, NU-23, ZSM-12, ZSM-22, ZSM-23, ZSM-35, ZSM-48, ZSM-50, ZSM-57, MeAPO-11, MeAPO-31, MeAPO-41, MgAPSO-11, MgAPSO-31, MgAPSO-41, MgAPSO-46, ELAPO-11, ELAPO-31, ELAPO-41, ELAPSO-11, ELAPSO-31, ELAPSO-41, laumontite, cancrinite, offretite, hydrogen form of stillbite, magnesium or calcium form of mordenite, and magnesium or calcium form of partheite, each of which may be used alone or in combination. ALPO-31 is described in U.S. Pat. No. 4,310,440. SAPO-11, SAPO-31, SAPO-37, and SAPO-41 are described in U.S. Pat. No. 4,440,871. SM-3 is described in U.S. Pat. Nos. 4,943,424; 5,087,347; 5,158,665; and 5,208,005. MgAPSO is a MeAPSO, which is an acronym for a metal aluminumsilicophosphate molecular sieve, where the metal Me is magnesium (Mg). Suitable MgAPSO-31 catalysts include MgAPSO-31. MeAPSOs are described in U.S. Pat. No. 4,793,984, and MgAPSOs are described in U.S. Pat. No. 4,758,419. MgAPSO-31 is an exemplary MgAPSO, where 31 means an MgAPSO having structure type 31.

In alternative embodiments, natural zeolites, such as ferrierite, are used, and these can have an initially reduced pore size that can be converted to forms suitable for olefin skeletal isomerization by removing associated alkali metal or alkaline earth metal by ammonium ion exchange and calcination to produce the substantially hydrogen form, as taught for example in U.S. Pat. Nos. 4,795,623 and 4,924,027. Further catalysts and conditions for skeletal isomerization can be used such as those disclosed in U.S. Pat. Nos. 5,510,306, 5,082,956, and 5,741,759.

In alternative embodiments, isomerization catalyst used in processes as provided herein also comprise a modifier selected from lanthanum, cerium, praseodymium, neodymium, samarium, gadolinium, terbium, and mixtures thereof, as described in U.S. Pat. Nos. 5,716,897 and 5,851,949. Other suitable support materials used in processes as provided herein include ZSM-22, ZSM-23, and ZSM-35, which are described for use in dewaxing in U.S. Pat. No. 5,246,566 and in the article entitled “New molecular sieve process for lube dewaxing by wax isomerization,” written by S. J. Miller, in Microporous Materials 2 (1994) 439-449.

In alternative embodiments, processes as provided herein also comprise use of processes as described in U.S. Pat. Nos. 4,310,440; 4,440,871; 4,793,984; 4,758,419; 4,943,424; 5,087,347; 5,158,665; 5,208,005; 5,246,566; 5,716,897; 5,851,949; 5,444,032 and 5,608,968, which teach for example that a suitable bifunctional catalyst which is constituted by an amorphous silica-alumina gel and one or more metals belonging to Group VIII, and which is effective in the hydro-isomerization of long-chain normal paraffins containing more than 15 carbon atoms.

In alternative embodiments, processes as provided herein comprise use of an activated carbon catalyst support, for example, as described in U.S. Pat. Nos. 5,981,419 and 5,908,134, which describe a suitable bifunctional catalyst which comprises: a porous crystalline material isostructural with beta-zeolite selected from boro-silicate (BOR—B) and boro-alumino-silicate (Al—BOR—B) in which the molar SiO₂:Al₂O₃ ratio is higher than 300:1; and, one or more metal(s) belonging to Group VIIIA, selected from platinum and palladium, in an amount comprised within the range of from 0.05 to 5% by weight. In alternative embodiments, processes as provided herein comprise use of processes as described in Article V. Calemma et al., App. Catal. A: Gen., 190 (2000), 207 teaches yet another suitable catalyst.

In alternative embodiments, the isomerization catalyst may be any of those well known in the art such as those described and cited above. In alternative embodiments, isomerization conditions include a temperature of about 150° C. to about 360° C. and a pressure of about 1724 kPa absolute (250 psia) to about 10342 kPa absolute (1500 psia). In another embodiment the isomerization conditions include a temperature of about 300° C. to about 360° C. and a pressure of about 3102 kPa absolute (450 psia) to about 6895 kPa absolute (1000 psia). In alternative embodiments, other operating conditions for the isomerization zone well known in the art are used.

In alternative embodiments, processes as provided herein operate at low pressures, which allows for the optional introduction of hydrogen from another unit, such as a hydrogen plant, without the use of a make-up compressor which may be an option to reduce or eliminate hydrogen recycle. When hydrogen is not recycled, the amount of hydrogen introduced to the isomerization zone would be only slightly greater than that which is consumed, for example, an excess of about 5 to about 25 percent of the consumption requirements.

In alternative embodiments, the final effluent stream, for example, the stream obtained after all reactions have been carried out, is now processed through one or more separation steps to obtain a purified hydrocarbon stream useful as a transportation fuel. With the final effluent stream comprising both a liquid component and a gaseous component, various portions of which are to be recycled, multiple separation steps may be employed. For example, hydrogen may be first separated in an isomerization effluent separator with the separated hydrogen being removed in an overhead stream. In alternative embodiments, suitable operating conditions of the isomerization effluent separator include, for example, a temperature of 230° C. and a pressure of 4100 kPa absolute (600 psia).

In alternative embodiments, if there is a low concentration of carbon oxides, or the carbon oxides are removed, the hydrogen may be recycled back to the hot high pressure hydrogen stripper for use both as a stripping gas and to combine with the remainder as a bottoms stream. In alternative embodiments, the remainder is passed to the isomerization reaction zone, and the hydrogen becomes a component of the isomerization reaction zone feed streams in order to provide the necessary hydrogen partial pressures for the reactor. In alternative embodiments, the hydrogen is also a reactant in the deoxygenation reactors, and different feedstocks will consume different amounts of hydrogen. Thus, in alternative embodiments, use of an isomerization effluent separator allows flexibility for the process to operate even when larger amounts of hydrogen are consumed in the first reaction zone. Furthermore, at least a portion of the remainder or bottoms stream of the isomerization effluent separator may be recycled to the isomerization reaction zone to increase the degree of isomerization.

In alternative embodiments, the remainder of the final effluent after the removal of hydrogen still has liquid and gaseous components and is cooled by techniques such as air cooling or water cooling, and passed to a cold separator where the liquid component is separated from the gaseous component. Suitable operating conditions of the cold separator include, for example, a temperature of about 20° C. to 60° C. and a pressure of 3850 kPa absolute (560 psia). In alternative embodiments, a water byproduct stream is also separated. At least a portion of the liquid component, after cooling and separating from the gaseous component, may be recycled back to the isomerization zone to increase the degree of isomerization. Prior to entering the cold separator, the remainder of the final effluent stream may be combined with the hot high pressure hydrogen stripper overhead stream, and the resulting combined stream may be introduced into the cold separator.

In alternative embodiments, the liquid component contains or comprises the hydrocarbons useful as transportation fuel, termed fuel range hydrocarbons, as well as smaller amounts of naphtha and LPG. The separated liquid component may be recovered as diesel fuel, or it may be further purified in a product stripper which separates lower boiling components and dissolved gases into an LPG and naphtha stream from the jet fuel and diesel fuel products containing C₈ to C₂₄ normal and branched alkanes. In alternative embodiments, suitable operating conditions of the product stripper include a temperature of from about 20° C. to about 200° C. at the overhead, and a pressure from about 0 to about 1379 kPa absolute (0 to 200 psia).

In alternative embodiments, the LPG and naphtha stream are further separated in a debutanizer or depropanizer in order to separate the LPG into an overhead stream, leaving the naphtha in a bottoms stream. Suitable operating conditions of this unit include or comprise a temperature of from about 20° C. to about 200° C. at the overhead, and a pressure from about 0 to about 2758 kPa absolute (0 to 400 psia). The LPG may be sold as valuable product, or it may be used in other processes such as a feed to a hydrogen production facility. Similarly, the naphtha may be used in other processes, such as the feed to a hydrogen production facility, a co-feed to a reforming process, or it may be used as a fuel blending component in the gasoline blending pool, for example.

In alternative embodiments, the gaseous component separated in the product separator comprises mostly hydrogen, and the carbon dioxide from the decarboxylation reaction. Other components such as carbon monoxide, propane, and hydrogen sulfide or other sulfur containing component may be present as well, and in alternative embodiments, they are separated and harvested.

It is desirable to recycle the hydrogen to the isomerization zone, but if the carbon dioxide was not removed, its concentration would quickly build up and effect the operation of the isomerization zone. Thus, in alternative embodiments, carbon dioxide is removed from the hydrogen by means well known in the art, such as for example reaction with a hot carbonate solution, pressure swing absorption, and the like. If desired, essentially pure carbon dioxide can be recovered by regenerating the spent absorption media.

In alternative embodiments, a sulfur containing component such as hydrogen sulfide is added to maintain the sulfided state of the deoxygenation catalyst or to control the relative amounts of the decarboxylation reaction and the hydrogenation reaction that are both occurring in the deoxygenation zone. In alternative embodiments, the amount of sulfur is controlled, and it can be removed before the hydrogen is recycled. The sulfur components may be removed using techniques such as absorption with an amine or by caustic wash. In alternative embodiments, depending upon the technique used, the carbon dioxide and sulfur containing components, and other components, are removed in a single separation step such as a hydrogen selective membrane.

In alternative embodiments, the hydrogen remaining after the removal of at least carbon dioxide is recycled to the reaction zone where hydrogenation primarily occurs and/or to any subsequent beds or reactors. In alternative embodiments, the recycle stream is introduced to the inlet of the reaction zone and/or to any subsequent beds or reactors.

One benefit of the hydrocarbon recycle is to control the temperature rise across the individual beds; thus, in alternative embodiments, the amount of hydrocarbon recycle may be determined based upon the desired hydrogen solubility in the reaction zone which is in excess of that used for temperature control. Increasing the hydrogen solubility in the reaction mixture allows for successful operation at lower pressures, and thus reduced cost.

In alternative embodiments, an exemplary hydrodeoxygenation and isomerization zones process as described herein comprises:

In alternative embodiments, the hydroprocessing and recovery process is first described in general with reference to FIG. 1. It is then described in more detail with reference to FIG. 2.

Turning to FIG. 1, the free fatty stream substantially free of glycerol, water, and metals 102 enters deoxygenation reaction zone 104 along with recycle hydrogen 126. Deoxygenated product 106 is stripped in hot high pressure hydrogen stripper 108 using hydrogen 114 a. The carbon oxides and water vapor are removed with hydrogen in overhead 110. Selectively stripped deoxygenated product 112 is passed to isomerization zone 116 along with recycle hydrogen 126 a and make-up hydrogen 114 b. Isomerized product 118 is combined with overhead 110 and passed to product recovery zone 120.

Carbon oxide stream 128, light ends stream 130, water byproduct stream 124, hydrogen stream 126, and branched paraffin-rich product 122 are removed from product recover zone 120. Branched paraffin-rich product 122 may be collected for use as transportation fuel, and hydrogen stream 126 is recycled to the deoxygenation reaction zone 104.

Turning to FIG. 2, the process begins with the free fatty stream substantially free of glycerol, water, and metals 2 which may pass through an optional feed surge drum. The feedstock stream 2 is combined with recycle gas stream 68 and recycle stream 16 to form combined feed stream 20, which is heat exchanged with reactor effluent and then introduced into deoxygenation reactor 4. The heat exchange may occur before or after the recycle is combined with the feed. Deoxygenation reactor 4 may contain multiple beds shown in FIG. 2 as 4 a, 4 b and 4 c.

In this exemplary embodiment, the deoxygenation reactor 4 contains at least one catalyst capable of catalyzing hydrodeoxygenation of the feedstock to remove oxygen. Deoxygenation reactor effluent stream 6 containing the products of the r hydrodeoxygenation reactions is removed from deoxygenation reactor 4 and heat exchanged with stream 20 containing feed to the deoxygenation reactor 4. Stream 6 comprises a liquid component containing largely normal paraffin hydrocarbons in the diesel boiling range and a gaseous component comprising hydrogen and vaporous water with minor amounts of carbon monoxide, carbon dioxide and light hydrocarbon gases.

In this exemplary embodiment, a deoxygenation reactor effluent stream 6 is then directed to hot high pressure hydrogen stripper 8. Make up hydrogen in line 10 is divided into two portions, streams 10 a and 10 b. Make up hydrogen in stream 10 a is also introduced to hot high pressure hydrogen stripper 8. In hot high pressure hydrogen stripper 8, the gaseous component of deoxygenation reactor effluent 6 is selectively stripped from the liquid component of deoxygenation reactor effluent 6 using make-up hydrogen 10 a and recycle hydrogen 28. The dissolved gaseous component comprising hydrogen, vaporous water, carbon monoxide, and carbon dioxide is selectively separated into hot high pressure hydrogen stripper overhead stream 14. The remaining liquid component of deoxygenation reactor effluent 6 comprising primarily normal paraffins having a carbon number from about 8 to about 24 with a cetane number of about 60 to about 100 is removed as hot high pressure hydrogen stripper bottom 12.

In this exemplary embodiment, a portion of hot high pressure hydrogen stripper bottoms forms recycle stream 16 and is combined with renewable feedstock stream 2 to create combined feed 20. Another portion of recycle stream 16, optional stream 16 a, may be routed directly to deoxygenation reactor 4 and introduced at interstage locations such as between beds 4 a and 4 b and/or between beds 4 b and 4 c to aid in temperature control, for example. The remainder of hot high pressure hydrogen stripper bottoms in stream 12 is combined with hydrogen stream 10 b to form combined stream 18 which is routed to isomerization reactor 22. Stream 18 may be heat exchanged with isomerization reactor effluent 24.

In this exemplary embodiment, the product of the isomerization reactor containing a gaseous portion of hydrogen and propane and a branched-paraffin-rich liquid portion is removed in line 24, and after optional heat exchange with stream 18, is introduced into hydrogen separator 26. The overhead stream 28 from hydrogen separator 26 contains primarily hydrogen which may be recycled back to hot high pressure hydrogen stripper 8. Bottom stream 30 from hydrogen separator 26 is air cooled using air cooler 32 and introduced into product separator 34. In product separator 34, the gaseous portion of the stream comprising hydrogen, carbon monoxide, hydrogen sulfide, carbon dioxide and light hydrocarbons are removed in stream 36 while the liquid hydrocarbon portion of the stream is removed in stream 38. A water byproduct stream 40 may also be removed from product separator 34. Stream 38 is introduced to product stripper 42 where components having higher relative volatilities are separated into stream 44 with the remainder, the paraffin components, being withdrawn from product stripper 42 in line 46. Stream 44 is introduced into fractionator 48 which operates to separate butane and lighter hydrocarbons into overhead 50 leaving a naphtha bottoms 52. Any of optional lines 72, 74, or 76 may be used to recycle at least a portion of the isomerization zone effluent back to the isomerization zone to increase the amount of n-paraffins that are isomerized to branched paraffins.

In this exemplary embodiment, the vapor stream 36 from product separator 34 contains the gaseous portion of the isomerization effluent which comprises at least hydrogen, carbon monoxide, hydrogen sulfide, and carbon dioxide and is directed to a system of amine absorbers to separate carbon dioxide and hydrogen sulfide from the vapor stream. Because of the cost of hydrogen, it is desirable to recycle the hydrogen to deoxygenation reactor 4, but it is not desirable to circulate the carbon dioxide or an excess of sulfur containing components. In order to separate sulfur containing components and carbon dioxide from the hydrogen, vapor stream 36 is passed through an amine absorber, also called a scrubber, in zone 56. The amine chosen to be employed in amine scrubber 56 is capable of selectively removing at least carbon dioxide. Suitable amines are available from for example DOW and from BASF, and in one embodiment the amines are a promoted or activated methyldiethanolamine (MDEA), or as described in U.S. Pat. No. 6,337,059. Suitable amines for the first amine absorber zone can be for example, from DOW, an can include the UCARSOL™ AP series solvents such as for example AP802, AP804, AP806, AP810 and AP814.

In this exemplary embodiment, the carbon dioxide is absorbed by the amine, while the hydrogen passes through the amine scrubber zone and into line 68 to be recycled to the first reaction zone. The amine is regenerated, and the carbon dioxide is released and removed in line 62. Within the amine absorber zone, regenerated amine may be recycled for use again. Conditions for the first scrubber zone include a temperature in the range of 30 to 60° C. The first absorber is operated at essentially the same pressure as the reaction zone. By “essentially” it is meant that the operating pressure of the first absorber is within about 1034 kPa absolute (150 psia) of the operating pressure of the reaction zone. For example, the pressure of the absorber is no more than 1034 kPa absolute (150 psia) less than that of the reaction zone.

In one aspect, the conversion of the glycerol and water recovered from the integrated hydrolysis step is achieved with high selectivity and low pressure. In alternative embodiments, a process for converting glycerol to substantially propylene glycol with high selectivity commences with providing a glycerol-containing material that has 50% or less by weight water. In alternative embodiments, this material is generated from the integrated hydrolysis step, whereby the glycerol is substantially removed from the free fatty acids generated, which are sent to the integrated deoxygenation reactor. In alternative embodiments, the glycerol and water containing material is contacted with hydrogen and a catalyst that is capable of hydrogenating glycerol, in order to form a reaction mixture. In alternative embodiments, conditions for reaction of the reaction mixture are established to include a temperature within a range from 150° C. to 250° C. and a pressure within a range from 0.1 bar to 25 bar. The reaction mixture is reacted under the conditions for reaction to first dehydrate the glycerol with resultant formation of acetol and second to hydrogenate the acetol to form propylene glycol as a reaction product. The reaction may be performed at temperatures of up to 270° C., 280° C. or even 290° C. or 300° C.; however, the use of these increased temperature results in thermal degradation of the reaction product together with die-reactions, and so is not recommended for applications where high purity of the reaction product is required. Thus, in alternative embodiments, processes comprise inclusion of increased amounts of water in the reagent stream facilitates improved selectivity. In alternative embodiments, by use of this process a yield of propylene glycol can be between about 90% to 98% pure, or the yield can be better between about 85% and 95%. The lower end of this range can be at least 150° C. to fully activate the catalyst and accelerate the reaction, but in some embodiments it is feasible to conduct the reaction in the range less than about 150° C.

In various other aspects, the glycerol-containing feedstock comprises from between about 5% to 50% water by weight. The catalyst may be a heterogenous catalyst that contains at least one element from Groups I or VIII of the Periodic Table. The catalyst may be a heterogeneous catalyst including at least one material selected from the group consisting of palladium, nickel, rhodium, copper, zinc, chromium and combinations thereof. The dehydration/hydrogenation catalyst may, for example, contain from between about 5 wt % to 95 wt % chromium, and may be comprised of compositions of copper expressed as CuO and chromium expressed as Cr₂O₃ at 30-80 wt % of CuO and 20-60 wt % of Cr₂O₃. In one example, the catalyst may be expressed as Cr₂O₃ at 40-60 wt % of CuO and 40-50 wt % of Cr₂O₃.

In alternative embodiments, a gas phase reaction is performed for converting glycerol to a product at high selectivity to propylene glycol and low selectivity to ethylene glycol. In alternative embodiments, the reaction commences with providing a gas phase reaction mixture that is essentially free of liquid and contains: glycerol with a partial pressure of glycerol in a range from 0.01 bars and 0.5 bars of glycerol, and hydrogen with a partial pressure of hydrogen between 0.01 and 25 bars of hydrogen. In alternative embodiments, the reaction mixture is maintained at a total pressure between 0.02 and 25 bars and contacts a heterogeneous catalyst at a temperature between 150° C. and 280° C. to form propylene glycol.

In alternative embodiments, in the gas phase reaction, a partial pressure of glycerol is preferably less than glycerol's dew point partial pressure in the reaction mixture, and greater than one fourth the dew point partial pressure in the reaction mixture. In alternative embodiments, this partial pressure is also preferably greater than half the dew point partial pressure in the reaction mixture. In alternative embodiments, the gas phase reaction mixture contains essentially no liquid and has a partial pressure of glycerol between 0.01 and 0.5 bars of glycerol and a partial pressure of hydrogen between 0.01 and 5 bars of hydrogen; and the reaction may be performed at a temperature between 150° C. and 280° C. to facilitate a reaction by use of the same catalysts described above or a mixture of catalyst beds whereby a catalyst to promote dehydration of glycerol is used in a first catalyst bed and a catalyst to promote hydrogenation is used in a second, third or any number of subsequent catalyst beds. In alternative embodiments, the total pressure of reaction may be between 0.02 and 5 bars.

In alternative embodiments, one method of preparing a reaction mixture comprising acetol and propylene glycol from glycerol includes a gas phase reaction at a temperature ranging from 1500 to 280° C. in a packed-bed reactor. In some embodiments this temperature is more preferably from 180° C. to 240° C. or 220° C. to avoid thermal degradation of reaction products. The reactions described herein occurred in a packed-bed reactor. In alternative embodiments, the pressures in the reaction vessel are from 0.01 to 50 bars, or from between 0.02 and 10 bars, or the reaction pressure exists within a range from 5 and 10, 20, 25, 30, 35 or 40 bars.

In alternative embodiments, water is added or water is generated as a reaction byproduct, and the water may be kept with the propylene glycol product or removed. In alternative embodiments, a major advantage of the current process is the very low concentration or absence of ethylene glycol resultant from either the use of copper chromite catalyst or formation and purification of acetol as an intermediate.

In one aspect of the invention, since glycerol has a vapor pressure of a mere 0.15 bar at 230° C., the hydrogen overpressure can add to this pressure to increase overall pressure—but this is primarily possible if glycerol is evaporated in the presence of a gas like hydrogen. In alternative embodiments, a condenser condenses the acetol and propylene glycol from unreacted gas; and although the unreacted gas may be purged, a recycle loop may be used to resupply the evaporator, packed bed reactor, or condenser by selective arrangement of valves such as a by-pass valve which allows for accurate dew point control of the mixture.

In various other aspects, the glycerol-containing feedstock comprises from between about 15% to 50% water by weight, optionally at a molar ratio of 3:1 water to glycerol. The catalyst may be a heterogenous catalyst that contains at least one element from Groups I or VIII of the Periodic Table. The catalyst may be a heterogeneous catalyst including at least one material selected from the group consisting of palladium, nickel, rhodium, copper, zinc, chromium and combinations thereof. The catalyst may, for example, contain from 5 wt % to 95 wt %, or 1 wt % to 98 wt %, chromium, and may be comprised of compositions of copper expressed as CuO and chromium expressed as Cr₂O₃ at 30-80 wt % of CuO and 20-60 wt % of Cr₂O₃. In one example, the catalyst may be expressed as Cr₂O₃ at 40-60 wt % of CuO and 40-50 wt % of Cr₂O₃. The presence of hydrogen reduces these oxides with their reduced form which is the active form of the catalyst for hydrogenation of acetol. The reaction may persist for a duration in a gas phase with reaction limited by catalyst within a range from 0.01 hour to 96 hours, such as from 4 to 46 hours or from 4 to 28 hours. In alternative embodiments, it is possible to operate the reaction at higher catalyst loadings in a gas phase with much shorter reaction times within the range from 0.001 to 8 hours, or more-preferably 0.002 to 1 hour, or even more preferably from 0.05 to 0.5 hours.

In alternative embodiments, a gas phase reaction is performed for converting glycerol to a product at high selectivity to propylene glycol and low selectivity to ethylene glycol. In alternative embodiments, the reaction commences with providing a gas phase reaction mixture that is essentially free of liquid and contains: glycerol with a partial pressure of glycerol in a range from 0.01 bars and 0.5 bars of glycerol, and hydrogen with a partial pressure of hydrogen between 0.01 and 25 bars of hydrogen. In alternative embodiments, the reaction mixture is maintained at a total pressure between 0.02 and 25 bars and contacts a heterogeneous catalyst at a temperature between 150° C. and 280° C. to form propylene glycol.

In alternative embodiments, in the gas phase reaction, a partial pressure of glycerol is less than glycerol's dew point partial pressure in the reaction mixture, and greater than one fourth the dew point partial pressure in the reaction mixture. In alternative embodiments, the partial pressure is greater than half the dew point partial pressure in the reaction mixture. In alternative embodiments, the gas phase reaction mixture contains essentially no liquid and has a partial pressure of glycerol between 0.01 and 0.5 bars of glycerol and a partial pressure of hydrogen between 0.01 and 50 bars of hydrogen; and the reaction may be performed at a temperature between 150° C. and 280° C. to facilitate a reaction by use of the same catalysts described above. The total pressure of reaction may be between 0.02 and 5 bars.

In alternative embodiments, although a gas phase packed bed reactor is preferred, other suitable reactor types include batch reactors, slurry batch reactors, trickle bed reactors, and teabag reactors. One reactor for use with highly exothermic reactions comprised of an outer shell containing straight tubes or U-tubes with an orientation such that the U-end of the U-Tubes is facing upward. The shell has an upper removable head where catalyst is loaded between shell and tubes from the top by removing the upper head. An inert packing may be is placed in the lowest portion of the space between the shell and U-Tubes at a depth between 2 and 24 inches.

In alternative embodiments, these reaction conditions provide a number of performance advantages. Operating at temperatures less than 250° C. dramatically reduces the amount of unintended by-product formation, for example, where lower concentrations of water may be used without formation of polymers or oligomers. Furthermore, operation at temperatures near 220° C., as compared to near 300° C., provides an increased relative volatility of propylene glycol that facilitates an improved separation of propylene glycol from the glycerol reaction mixture. The use of lower pressures allows the use of less expensive reaction vessels, for example, as compared to high-pressure vessels that operate above about 28 bars, while also permitting the propylene glycol to distill from solution at these temperatures. Even so, some embodiments are not limited to use at pressures less than about 50 bars, and may in fact be practiced at very high hydrogen pressures. These exemplary process conditions are viable at lower pressures (less than 20 bar) whereas most other processes to produce similar products require much higher pressures.

In alternative embodiments the acid-washed brown grease is then hydrolyzed in the presence of an acid catalyst at subcritical conditions; for example, temperature of less than about 350° C. and a pressure of less than about 6895 kPa(g) (1000 psig), to convert about greater than (>) 80% of the total lipids present to FFA. Hydrolysis reaction time would be about 75 minutes.

In alternative embodiments, using the separation and purification processes described, a free fatty acid stream substantially free of glycerol, water, and metals (for example, having less than 10 wt-ppm metals, total glycerol less than (<) 0.5% by weight, and total water content less than (<) 1% by weight) is recovered from the catalytic hydrolysis reactor effluent. In alternative embodiments a glycerol-containing co-product is generated, or substantially isolated.

In alternative embodiments, the FFA stream substantially free of glycerol, water, and metals is pressurized, mixed with hydrogen gas, heated, and passed over a fixed bed of hydrodeoxygenation catalyst at a reactor partial pressure of 4826 kPa (a) (700 psia) to convert more than 95% of the carbon in the free fatty acid stream substantially free of glycerol, water, and metals to oxygenate-free hydrocarbons. Catalyst and reaction conditions can be selected to minimize feedstock carbon losses to carbon oxides through decarboxylation and decarbonylation reactions.

In alternative embodiments, a distillate comprising diesel fuel and jet fuel having lower heating values greater than (>) 40 MJ/kg is recovered.

In alternative embodiments, a stream comprising glycerol and water is purified using the separation and purification processes described to a glycerol stream substantially free of free fatty acids. The stream comprising glycerol and water substantially free of free fatty acids could be selectively hydrogenated in a hydrogenation zone under hydrogenation conditions (for example, a temperature of about 215° C. and a pressure of about 7 bar) in the presence of a hydrogenation catalyst. In alternative embodiments the process achieves about 98% conversion of glycerol, with about 95% selectivity to propylene glycol and 0.5% selectivity to ethylene glycol.

In alternative embodiments a feedstock of marine algae (for example, of the genus Nanochloropsis, or for example comprising N. australis, N. gaditana, N. granulata, N. limnetica, N. oceanica, N. oculata, and/or N. salina) with a dry basis neutral lipid content of 20% and a polar lipid content of 10% is pretreated with an aqueous acid stream to remove at least a portion of the metals and cellulosic materials.

In alternative embodiments a lipid-enriched fraction is recovered and then hydrolyzed in the presence of an acid catalyst at subcritical conditions to convert 98% of the neutral lipids and greater than (>) 50% of the polar lipids present to FFA. Hydrolysis reaction time would be about 90 minutes.

In alternative embodiments, using the separation and purification processes described, a free fatty acid stream substantially free of glycerol, water, and metals, (for example, having less than 10 wt-ppm metals, total glycerol less than (<) 0.5% by weight, and total water content less than (<) 1% by weight) is recovered from the catalytic hydrolysis reactor effluent, and a glycerol stream comprising, for example, less than (<) 5% wt of the total fatty acids present in the glyceride containing stream.

In alternative embodiments the free fatty acid stream substantially free of glycerol, water, and metals is then be pressurized, mixed with hydrogen gas, heated, and passed over a fixed bed of hydrodeoxygenation catalyst at a reactor partial pressure of 5516 kPa (a) (800 psia) to convert more than 90% of the carbon in the free fatty acid stream substantially free of glycerol, water, and metals to oxygenate-free hydrocarbons. Catalyst and reaction conditions can be selected to minimize feedstock carbon losses to carbon oxides through decarboxylation and decarbonylation reactions.

In alternative embodiments a distillate comprising diesel fuel and jet fuel having lower heating values greater than (>) 40 MJ/kg is recovered.

In alternative embodiments the stream comprising glycerol and water is purified using the separation and purification processes described to a glycerol stream substantially free of free fatty acids.

In alternative embodiments the stream comprising glycerol and water substantially free of free fatty acids is selectively hydrogenated in a hydrogenation zone under hydrogenation conditions (for example, a temperature of about 215° C. and a pressure of about 7 bar) in the presence of a hydrogenation catalyst. The process achieves about 98% conversion of glycerol, with about 95% selectivity to propylene glycol and 0.5% selectivity to ethylene glycol.

### Exemplary Processes

### Feedstocks Comprising Brown Grease

In alternative embodiments, a feedstock of brown grease with free fatty acid (FFA) to lipid weight ratio greater than (>) 0.3, a sulfur content over 500 wt-ppm and a metals (Na, Mg, Ca, Zn) content greater than (>) 1000 wt-ppm is acid-washed to reduce the metals concentration below 30 wt-ppm.

In alternative embodiments, the acid-washed brown grease is then hydrolyzed in the presence of an acid catalyst at subcritical conditions; for example, temperature of less than about 350° C. and a pressure of less than about 6895 kPa(g) (1000 psig), to convert about greater than (>) 80% of the total lipids present to FFA. Hydrolysis reaction time would be about 75 minutes.

In alternative embodiments, using the separation and purification processes described, a free fatty acid stream substantially free of glycerol, water, and metals (for example, having less than 10 wt-ppm metals, total glycerol less than (<) 0.5% by weight, and total water content less than (<) 1% by weight) can be recovered from the catalytic hydrolysis reactor effluent. A glycerol-containing co-product also can be generated.

In alternative embodiments, the FFA stream substantially free of glycerol, water, and metals is pressurized, mixed with hydrogen gas, heated, and passed over a fixed bed of hydrodeoxygenation catalyst at a reactor partial pressure of 4826 kPa (a) (700 psia) to convert more than 95% of the carbon in the free fatty acid stream substantially free of glycerol, water, and metals to oxygenate-free hydrocarbons. Catalyst and reaction conditions can be selected to minimize feedstock carbon losses to carbon oxides through decarboxylation and decarbonylation reactions.

In alternative embodiments, a distillate comprising diesel fuel and jet fuel having lower heating values greater than (>) 40 MJ/kg can be recovered.

In alternative embodiments, the stream comprising glycerol and water is purified using the separation and purification processes described to a glycerol stream substantially free of free fatty acids. In alternative embodiments, the stream comprising glycerol and water substantially free of free fatty acids is selectively hydrogenated in a hydrogenation zone under hydrogenation conditions (for example, a temperature of about 215° C. and a pressure of about 7 bar) in the presence of a hydrogenation catalyst. The process can achieve about 98% conversion of glycerol, with about 95% selectivity to propylene glycol and 0.5% selectivity to ethylene glycol.

### Feedstocks Comprising Marine Algae

In alternative embodiments, a feedstock of marine algae (for example, of the genus Nanochloropsis, or for example comprising N. australis, N. gaditana, N. granulata, N. limnetica, N. oceanica, N. oculata, and/or N. salina) with a dry basis neutral lipid content of 20% and a polar lipid content of 10% is pretreated with an aqueous acid stream to remove at least a portion of the metals and cellulosic materials.

In alternative embodiments, a lipid-enriched fraction is recovered and then hydrolyzed in the presence of an acid catalyst at subcritical conditions to convert 98% of the neutral lipids and greater than (>) 50% of the polar lipids present to FFA. Hydrolysis reaction time would be about 90 minutes.

In alternative embodiments, using the separation and purification processes described, a free fatty acid stream substantially free of glycerol, water, and metals, (for example, having less than 10 wt-ppm metals, total glycerol less than (<) 0.5% by weight, and total water content less than (<) 1% by weight) is recovered from the catalytic hydrolysis reactor effluent, and a glycerol stream comprising, for example, less than (<) 5% wt of the total fatty acids present in the glyceride containing stream.

In alternative embodiments, the free fatty acid stream substantially free of glycerol, water, and metals is then be pressurized, mixed with hydrogen gas, heated, and passed over a fixed bed of hydrodeoxygenation catalyst at a reactor partial pressure of 5516 kPa (a) (800 psia) to convert more than 90% of the carbon in the free fatty acid stream substantially free of glycerol, water, and metals to oxygenate-free hydrocarbons. In alternative embodiments, catalyst and reaction conditions are selected to minimize feedstock carbon losses to carbon oxides through decarboxylation and decarbonylation reactions.

In alternative embodiments, a distillate comprising diesel fuel and jet fuel having lower heating values greater than (>) 40 MJ/kg is recovered.

In alternative embodiments, the stream comprising glycerol and water is purified using the separation and purification processes described to a glycerol stream substantially free of free fatty acids.

In alternative embodiments, the stream comprising glycerol and water substantially free of free fatty acids is selectively hydrogenated in a hydrogenation zone under hydrogenation conditions (for example, a temperature of about 215° C. and a pressure of about 7 bar) in the presence of a hydrogenation catalyst. The process achieves about 98% conversion of glycerol, with about 95% selectivity to propylene glycol and 0.5% selectivity to ethylene glycol.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Integrated Hydrolysis Followed by Hydrodeoxygenation Process and Hydrodeoxygenation Process

This example demonstrates an exemplary process comprising integrated hydrolysis of feedstocks followed by a hydrodeoxygenation process with a stand-alone hydrodeoxygenation process.

As shown in FIG. 1, a pretreated renewable glyceride-rich feedstock 305 and water 310 enter a hydrolysis zone 315. The glycerides are hydrolyzed to FFAs and glycerol. The FFAs and glycerol hydrolysis products enter a separation zone 320. The FFAs and glycerol are separated into a free fatty acid stream substantially free of glycerol, water, and metals 325 and a glycerol containing stream 330. The separation zone 320 is described in more detail below.

The free fatty acid stream substantially free of glycerol, water, and metals 325 is sent to the hydroprocessing zone 335 where the FFAs are reacted with hydrogen from hydrogen source 340 to produce a paraffin product useful as a transportation fuel 345 which is sent to recovery zone 350 where the n-paraffin product 362 is recovered.

In alternative embodiments, or optionally, recycle water 355 from the separation zone 320 and/or the paraffin recovery zone 350 can be combined with make-up water to provide at least a portion of the water stream 310 for the hydrolysis zone 315.

The glycerol containing stream 330 and water 310 and or water 355 is sent to selective hydrogenation zone 360, where it is the glycerol and water are mixed and then reacted with hydrogen from hydrogen source 340 in the presence of a hydrogenation catalyst 365. The polyol product 370 is then recovered in polyol recovery zone 375 as polyol product 380.

One embodiment of a process for separating the hydrolysis products into the free fatty acid stream substantially free of glycerol, water, and metals and the glycerol-containing stream is illustrated in FIG. 2. The effluent 405 from the hydrolysis zone 400 enters extraction column 410. The extraction column 410 separates the product into a free fatty acid enriched stream 415 and a glycerol-containing stream 420. The free fatty acid enriched stream 415 is optionally sent to one or more purification devices 425, 430 to remove water, glycerol, and metals producing the free fatty acid stream substantially free of glycerol, water, and metals 435. The free fatty acid stream substantially free of glycerol, water, and metals 435 is sent to the hydroprocessing zone where it is reacted with hydrogen in the presence of a catalyst to hydrodeoxygenate it, producing n-paraffins and water.

As illustrated in FIG. 1, the free fatty stream substantially free of glycerol, water, and metals 102 enters deoxygenation reaction zone 104 along with recycle hydrogen 126. Deoxygenated product 106 is stripped in hot high pressure hydrogen stripper 108 using hydrogen 114 a. The carbon oxides and water vapor are removed with hydrogen in overhead 110. Selectively stripped deoxygenated product 112 is passed to isomerization zone 116 along with recycle hydrogen 126 a and make-up hydrogen 114 b. Isomerized product 118 is combined with overhead 110 and passed to product recovery zone 120. Carbon oxide stream 128, light ends stream 130, water byproduct stream 124, hydrogen stream 126, and branched paraffin-rich product 122 are removed from product recover zone 120. Branched paraffin-rich product 122 may be collected for use as transportation fuel, and hydrogen stream 126 is recycled to the deoxygenation reaction zone 104.

As illustrated in FIG. 2, the process begins with the free fatty stream substantially free of glycerol, water, and metals 2 which may pass through an optional feed surge drum. The feedstock stream 2 is combined with recycle gas stream 68 and recycle stream 16 to form combined feed stream 20, which is heat exchanged with reactor effluent and then introduced into deoxygenation reactor 4. The heat exchange may occur before or after the recycle is combined with the feed. Deoxygenation reactor 4 may contain multiple beds shown in FIG. 2 as 4 a, 4 b and 4 c.

In this exemplary embodiment, the deoxygenation reactor 4 contains at least one catalyst capable of catalyzing hydrodeoxygenation of the feedstock to remove oxygen. Deoxygenation reactor effluent stream 6 containing the products of the r hydrodeoxygenation reactions is removed from deoxygenation reactor 4 and heat exchanged with stream 20 containing feed to the deoxygenation reactor 4. Stream 6 comprises a liquid component containing largely normal paraffin hydrocarbons in the diesel boiling range and a gaseous component comprising hydrogen and vaporous water with minor amounts of carbon monoxide, carbon dioxide and light hydrocarbon gases.

In this exemplary embodiment, a deoxygenation reactor effluent stream 6 is then directed to hot high pressure hydrogen stripper 8. Make up hydrogen in line 10 is divided into two portions, streams 10 a and 10 b. Make up hydrogen in stream 10 a is also introduced to hot high pressure hydrogen stripper 8. In hot high pressure hydrogen stripper 8, the gaseous component of deoxygenation reactor effluent 6 is selectively stripped from the liquid component of deoxygenation reactor effluent 6 using make-up hydrogen 10 a and recycle hydrogen 28. The dissolved gaseous component comprising hydrogen, vaporous water, carbon monoxide, and carbon dioxide is selectively separated into hot high pressure hydrogen stripper overhead stream 14. The remaining liquid component of deoxygenation reactor effluent 6 comprising primarily normal paraffins having a carbon number from about 8 to about 24 with a cetane number of about 60 to about 100 is removed as hot high pressure hydrogen stripper bottom 12.

In this exemplary embodiment, a portion of hot high pressure hydrogen stripper bottoms forms recycle stream 16 and is combined with renewable feedstock stream 2 to create combined feed 20. Another portion of recycle stream 16, optional stream 16 a, may be routed directly to deoxygenation reactor 4 and introduced at interstage locations such as between beds 4 a and 4 b and/or between beds 4 b and 4 c to aid in temperature control, for example. The remainder of hot high pressure hydrogen stripper bottoms in stream 12 is combined with hydrogen stream 10 b to form combined stream 18 which is routed to isomerization reactor 22. Stream 18 may be heat exchanged with isomerization reactor effluent 24.

In this exemplary embodiment, the product of the isomerization reactor containing a gaseous portion of hydrogen and propane and a branched-paraffin-rich liquid portion is removed in line 24, and after optional heat exchange with stream 18, is introduced into hydrogen separator 26. The overhead stream 28 from hydrogen separator 26 contains primarily hydrogen which may be recycled back to hot high pressure hydrogen stripper 8. Bottom stream 30 from hydrogen separator 26 is air cooled using air cooler 32 and introduced into product separator 34. In product separator 34, the gaseous portion of the stream comprising hydrogen, carbon monoxide, hydrogen sulfide, carbon dioxide and light hydrocarbons are removed in stream 36 while the liquid hydrocarbon portion of the stream is removed in stream 38. A water byproduct stream 40 may also be removed from product separator 34. Stream 38 is introduced to product stripper 42 where components having higher relative volatilities are separated into stream 44 with the remainder, the paraffin components, being withdrawn from product stripper 42 in line 46. Stream 44 is introduced into fractionator 48 which operates to separate butane and lighter hydrocarbons into overhead 50 leaving a naphtha bottoms 52. Any of optional lines 72, 74, or 76 may be used to recycle at least a portion of the isomerization zone effluent back to the isomerization zone to increase the amount of n-paraffins that are isomerized to branched paraffins.

Table 1 provides a simplified comparison of this exemplary integrated hydrolysis followed by a hydrodeoxygenation process with a stand-alone hydrodeoxygenation process. Full conversion of feed oxygenate to water is assumed.

TABLE 1

| Hydrolysis/ | Mass | Hydrode- | Mass |
|---|---|---|---|
| Hydrodeoxygenation | Units | oxygenation | Units |
| C16 Triglyceride | 100.0 | C16 Triglyceride | 100.0 |
| H2O | 6.7 | | |
| Hydrolysis Products | | | |
| C16 FFA | 95.3 | | |
| Glycerol | 11.4 | | |
| | 106.7 | | |
| Hydrodeoxygenation Inputs | | | |
| C16 FFA | 95.3 | | |
| H2 | 2.2 | H2 | 3.0 |
| | 97.5 | | |
| Hydrodeoxygenation Products | | | |
| Propane | 0.0 | Propane | 5.5 |
| Diesel Range Paraffins | 84.1 | Diesel Range Paraffins | 84.1 |
| Water | 13.4 | Water | 13.4 |

### Example 2: Integrated Hydrolysis of Brown Grease Feedstock

This example demonstrates an exemplary process using brown grease feedstock, the process comprising integrated hydrolysis of the feedstock followed by a hydrodeoxygenation process with a stand-alone hydrodeoxygenation process.

As shown in Table 2, a feedstock of brown grease with FFA to lipid weight ratio greater than (>) 0.3, a sulfur content over 500 wt-ppm and a metals (Na, Mg, Ca, Zn) content greater than (>) 1000 wt-ppm was acid-washed reduced the metals concentration below 30 wt-ppm:

TABLE 2
Brown Grease Feedstock and Acid-Washed Product

| | S | Na | Mg | Ca | Zn |
|---|---|---|---|---|---|
| Brown grease as received | 640 | 37 | 61 | 1076 | 58 |
| HCl (aq) treated and | 428 | 4.3 | 1.5 | 13.1 | 7 |
| washed brown grease | | | | | |

In alternative embodiments the acid-washed brown grease is then hydrolyzed in the presence of an acid catalyst at subcritical conditions; for example, temperature of less than about 350° C. and a pressure of less than about 6895 kPa(g) (1000 psig), to convert about greater than (>) 80% of the total lipids present to FFA. Hydrolysis reaction time would be about 75 minutes.

In alternative embodiments, using the separation and purification processes described, a free fatty acid stream substantially free of glycerol, water, and metals (for example, having less than 10 wt-ppm metals, total glycerol less than (<) 0.5% by weight, and total water content less than (<) 1% by weight) is recovered from the catalytic hydrolysis reactor effluent. In alternative embodiments a glycerol-containing co-product is generated, or substantially isolated.

In alternative embodiments, the FFA stream substantially free of glycerol, water, and metals is pressurized, mixed with hydrogen gas, heated, and passed over a fixed bed of hydrodeoxygenation catalyst at a reactor partial pressure of 4826 kPa (a) (700 psia) to convert more than 95% of the carbon in the free fatty acid stream substantially free of glycerol, water, and metals to oxygenate-free hydrocarbons. Catalyst and reaction conditions can be selected to minimize feedstock carbon losses to carbon oxides through decarboxylation and decarbonylation reactions.

In alternative embodiments, a distillate comprising diesel fuel and jet fuel having lower heating values greater than (>) 40 MJ/kg is recovered.

In alternative embodiments, a stream comprising glycerol and water is purified using the separation and purification processes described to a glycerol stream substantially free of free fatty acids. The stream comprising glycerol and water substantially free of free fatty acids could be selectively hydrogenated in a hydrogenation zone under hydrogenation conditions (for example, a temperature of about 215° C. and a pressure of about 7 bar) in the presence of a hydrogenation catalyst. In alternative embodiments the process achieves about 98% conversion of glycerol, with about 95% selectivity to propylene glycol and 0.5% selectivity to ethylene glycol.

### Example 3: Integrated Hydrolysis of Marine Algae Feedstock

This example demonstrates an exemplary process using marine algae feedstock, the process comprising integrated hydrolysis of the feedstock followed by a hydrodeoxygenation process with a stand-alone hydrodeoxygenation process.

A feedstock of marine algae (for example, of the genus Nanochloropsis, or for example comprising N. australis, N. gaditana, N. granulata, N. limnetica, N. oceanica, N. oculata, and/or N. salina) with a dry basis neutral lipid content of 20% and a polar lipid content of 10% is pretreated with an aqueous acid stream to remove at least a portion of the metals and cellulosic materials.

A lipid-enriched fraction is recovered and then hydrolyzed in the presence of an acid catalyst at subcritical conditions to convert 98% of the neutral lipids and greater than (>) 50% of the polar lipids present to FFA. Hydrolysis reaction time would be about 90 minutes.

Using the separation and purification processes described, a free fatty acid stream substantially free of glycerol, water, and metals, (for example, having less than 10 wt-ppm metals, total glycerol less than (<) 0.5% by weight, and total water content less than (<) 1% by weight) is recovered from the catalytic hydrolysis reactor effluent, and a glycerol stream comprising, for example, less than (<) 5% wt of the total fatty acids present in the glyceride containing stream.

The free fatty acid stream substantially free of glycerol, water, and metals is then be pressurized, mixed with hydrogen gas, heated, and passed over a fixed bed of hydrodeoxygenation catalyst at a reactor partial pressure of 5516 kPa (a) (800 psia) to convert more than 90% of the carbon in the free fatty acid stream substantially free of glycerol, water, and metals to oxygenate-free hydrocarbons. Catalyst and reaction conditions can be selected to minimize feedstock carbon losses to carbon oxides through decarboxylation and decarbonylation reactions.

A distillate comprising diesel fuel and jet fuel having lower heating values greater than (>) 40 MJ/kg is recovered.

The stream comprising glycerol and water is purified using the separation and purification processes described to a glycerol stream substantially free of free fatty acids.

The stream comprising glycerol and water substantially free of free fatty acids is selectively hydrogenated in a hydrogenation zone under hydrogenation conditions (for example, a temperature of about 215° C. and a pressure of about 7 bar) in the presence of a hydrogenation catalyst. The process achieves about 98% conversion of glycerol, with about 95% selectivity to propylene glycol and 0.5% selectivity to ethylene glycol.

A number of embodiments of the invention have been described.

Nevertheless, it can be understood that various modifications may be made without departing from the spirit and scope of the invention. Accordingly, other embodiments are within the scope of the following claims.

1: An integrated process for producing paraffins and polyols from a glyceride-comprising renewable feedstock.
wherein optionally the paraffin comprises a C8 to C18 linear alkane and/or a branched alkane, and optionally the polyol comprises 1,2 propanediol (1,2 PDO); 1,3 PDO; ethylene glycol (or ethane-1,2-diol); 1,2 butanediol (BDO); 2,3 BDO and/or 1,4 BDO,
the method comprising:
(a) reacting the glyceride-comprising renewable feedstock with water in a first reaction zone to produce an effluent stream comprising a plurality of free fatty acids (FFAs), glycerol, and water,
wherein optionally the reacting comprises reaction conditions comprising: a temperature in the range of between about 150° C. to 250° C. or 100° C. to 300° C., pressure at between about 300 psi to 500 psi (1 MPa is 145.038 psi (pound force per square inch)), and/or a reaction time of between about 0.1 hours (hrs) to 5 hrs;
(b) separating or substantially separating the free fatty acids from the glycerol and water to produce a free fatty acid (FFA) stream substantially free of glycerol, water, and metals, and a glycerol and water stream substantially free of FFAs;
(c) reacting the free fatty acid stream substantially free of glycerol, water, and metals with hydrogen from a hydrogen source in a second reaction zone in the presence of a hydroprocessing catalyst under hydroprocessing conditions thereby hydrodeoxygenating the free fatty acid stream substantially free of glycerol, water, and metals to produce a reaction product comprising n-paraffins and water,
wherein optionally the hydroprocessing conditions comprise: a temperature in the range of between about 200° C. to 500° C., or between about 280° C. to 450° C.;
and/or a pressure of between about 1 to 5 MPa (megapascal) (1 MPa is 145.038 psi);
(d) recovering the n-paraffin reaction product;
(e) reacting the stream comprising glycerol and water that is substantially free of free fatty acids with hydrogen from the hydrogen source in a third reaction zone under hydrogenation conditions to produce a reaction product comprising polyols,
wherein optionally the hydrogenation conditions comprise: a temperature in the range of between about 190° C. to 350° C., or between about 100° C. to 500° C., or between about 200° C. to 300° C., or between about 210° C. to 220° C., or between about 280° C. to 450° C.; and/or a pressure of between about 0.5 to about 2 MPa or between about 1 to 5 MPa or between about 0.1 to 1.5 MPa (megapascal); and
(e) recovering, removing or substantially isolating the polyol reaction product.

2: The integrated process of claim 1, further comprising contacting at least a portion of the n-paraffin reaction product with an isomerization catalyst under isomerization conditions to isomerize at least a portion of (or at least about 1%, 5%, or 10% of, or between 0.5% to 15% of, or between about 1% to 50% of) the n-paraffins to isoparaffins,
and optionally the isomerization conditions comprise between about 350° C. to 450° C. or 300° C. to 500° C., and/or a pressure of between about 3 MPa to 5 MPa, or between about 2 MPa to 7 MPa.

3: The integrated process of claim 1, wherein separating the free fatty acids from the glycerol and water comprises at least one process comprising: hot depressurization, cyclonic vapor liquid separation, liquid-liquid separation, multi-effect evaporation, steam distillation, vacuum distillation, reactive distillation, liquid extraction, adsorption, filtration, membrane separation, absorption and/or ion exchange.

4: The integrated process of claim 1, wherein separating or substantially separating the free fatty acids from the glycerol and water comprises:
separating or substantially separating the effluent stream into a free fatty acid-enriched stream and a glycerol-comprising stream; and
purifying or substantially purifying the free fatty acid enriched stream to form the free fatty acid stream substantially free of glycerol, water, and metals.

5: The integrated process of claim 4, further comprising purifying or substantially purifying the glycerol containing stream to form the glycerol and water stream substantially free of free fatty acids.

6: The integrated process of claim 1, wherein the free fatty acid stream substantially free of glycerol, water, and metals has less than about 5 wt % glycerol, less than about 5 wt % water, and less than about 20 ppm (wt) metals.

7: The integrated process of claim 1, wherein reacting the glyceride-comprising renewable feedstock with water takes place in the presence of a hydrolysis catalyst.

8: The integrated process of claim 1, wherein reacting the glyceride-comprising renewable feedstock with water is performed at sub-critical conditions,
and optionally the subcritical conditions comprise pressure of less than about 373° C. and 220 bars (1 MPa is equivalent to 10 bar).

9: The integrated process of claim 1, wherein reacting the glyceride-comprising renewable feedstock with water takes place at a pressure of less than about 5516 kPa(g) and a temperature of less than about 300° C.

10: The integrated process of claim 1, wherein the glyceride-comprising renewable feedstock comprises algae or a stream derived from algae.

11: The integrated process of claim 1, further comprising contacting the glyceride-comprising renewable feedstock with one or more of an acid, a base, an extractive material, an adsorptive material or any combination thereof to remove or substantially remove contaminants from the glyceride-comprising renewable feedstock before reacting the glyceride-comprising renewable feedstock with the water.

12: The integrated process of claim 1, wherein the hydroprocessing conditions comprise a temperature of less than about 400° C., a hydrogen partial pressure of greater than about 3450 kPa, an H₂ to organic oxygen ratio greater than about 5, and in the presence of a base metal catalyst.

13: The integrated process of claim 1, wherein at least about 80 wt % of the total lipids in the glyceride-comprising renewable feedstock are converted to free fatty acids in the reaction with water.

14: The integrated process of claim 1, wherein when reacting the glyceride-comprising renewable feedstock with water, a ratio of water to total lipids in the first reaction zone is greater than a stoichiometric minimum required for complete hydrolysis.

15: The integrated process of claim 1, wherein at least about 80 wt % of oxygen in the free fatty acid stream substantially free of glycerol, water, and metals is converted to water and less than 10% of carbon in the free fatty acid stream substantially free of glycerol, water, and metals is reacted to form CO or CO₂ in the second reaction zone.

16: The integrated process of claim 1, wherein at least about 80% of the n-paraffins in the reaction product have an even number of carbon atoms.

17: The integrated process of claim 1, wherein reacting the free fatty acid stream substantially free of glycerol, water, and metals with hydrogen comprises selective catalytic hydrodeoxygenation.

18: The integrated process of claim 1, wherein the third reaction zone the gas phase reaction mixture includes hydrogen at a partial pressure of hydrogen in a range from 0.01 and 25 bars of hydrogen.

19: The integrated process of claim 1, wherein:
(a) in the third reaction zone the gas phase mixture of glycerol and water is contacted with a heterogeneous catalyst that comprises at least one element from Groups I or VIII of the Periodic Table, ruthenium, copper, chromite, nickel and combinations thereof;
(b) the third reaction zone the gas phase mixture of glycerol, hydrogen and water comprises essentially or substantially no liquid and has a partial pressure of glycerol between about 0.01 and about 0.5 bars of glycerol; and establishing a temperature in a range from about 80° C. to about 300° C. to facilitate a reaction;
(c) the third reaction zone the gas phase mixture of glycerol, hydrogen and water comprises between about 0.05% to 5% water, or between about 1% to 2% water, or between about 1% to 50% water, or about 1%, 1.5%, 2%, 5%, 10% or 15% water;
(d) the glyceride-comprising renewable feedstock is comprised of or derived from a microbial, a plant and/or an animal material or source;
and optionally the microbial, plant and/or animal material or source comprises a microbial, a plant and/or an animal oil, optionally a corn, jatropha, camelina, rapeseed, canola, soybean and/or algal oil, or canola oil, corn oil, soy oils, rapeseed oil, soybean oil, colza oil, tall oil, sunflower oil, hempseed oil, olive oil, linseed oil, coconut oil, castor oil, peanut oil, pennycress oil, palm oil, carinata oil, jojoba oil, mustard oil, cottonseed oil, jatropha oil, tallow, yellow and brown greases, lard, train oil, fats in milk, fish oil, algal oil, sewage sludge, or an animal fat, wherein optionally the animal fat comprises or is derived from a tallow, a fish oil, a waste streams, a yellow and/or a brown grease, an oily stream recovered from sewage sludge, or any combination thereof; and/or
(e) the glyceride in the glyceride-comprising renewable feedstock comprises mono- di- and/or tri-glycerides.

20-24. (canceled)

25: An industrial plant for integrated hydrolysis/hydroprocessing processing comprising components for practicing a process of claim 1, and optionally the industrial plant comprises components as described in FIG. 1
